(19) United States
(12) Patent Application Publication
(10) Pub. No.: US 2025/0085278 A1
(43) Pub. Date: Mar. 13, 2025

(54) LATERAL FLOW ASSAY BY USING CARBOXYL LATEX BEADS AND BIOTIN-POLYSTREPTAVIDIN FOR THE DETECTION OF COVID-19 INFECTION AND DIAGNOSTIC KIT USING THE LATERAL FLOW ASSAY

(71) Applicants: Jung Joo Moon, Newark, NJ (US); Sung Woo Moon, Seoul (KR)

(72) Inventors: Jung Joo Moon, Newark, NJ (US); Sung Woo Moon, Seoul (KR)

(21) Appl. No.: 18/686,412

(22) PCT Filed: Aug. 25, 2022
(86) PCT No.: PCT/US2022/075473

§ 371 Date: Feb. 24, 2024

Related U.S. Application Data
(60) Provisional application No. 63/237,076, filed on Aug. 25, 2021.
(60) Provisional application No. 63/311,050, filed on Feb. 17, 2022.

(51) Int. Cl. G01N 33/543; G01N 33/569; G01N 33/58

(52) U.S. Cl. CPC G01N 33/54388; G01N 33/54313; G01N 33/56983; G01N 33/585; G01N 2333/165; G01N 2333/46

(57) ABSTRACT

The systems and methods herein are directed to carboxylated latex beads and biotin-polysterptavidin as a label material for fluorescence lateral flow assay (LFA) for the diagnosis of SARS-Cov-9 with increased sensitivity. The carboxylated latex and biotin-polystreptavidin instantly increases the fluorescence intensity and the resulting signal enhancement significantly increases sensitivity for analyte detection. The LFA can also be used for the detection of SARS-Cov-2 and as rapid testing kit of COVID-19. The LFA kit allows detection of nucleocapsid protein of SARS-Cov-2 as little as 0.1 ng/ml and as a point of care testing of COVID-19.

### BACKGROUND

Many Immunoassays and genetic analysis are two of the mainstems of analytical methods used in modern microbiology to identify infectious agents in addition to previous methods such as culture. Immunoassays can provide a fast, simple and a cost-effective method of detection. Immunoassays have capacity for high throughput and numerous samples can be analyzed simultaneously, significantly reducing the average analytical time. Some immunoassays rely on host immunological responses to a given infectious agent, for instance, by testing for the presence of host antibodies that specifically bind to one or more unique antigens of that infectious agent, utilizing the antigen-antibody response. Numerous types of immunoassay systems are available for diagnostic purposes, including large, automated central lab systems and relatively simple over-the-counter tests. These immunoassays utilize a broad range of test formats, such as agglutination assays, precipitin assays, enzyme-linked immunoassays, direct fluorescence assays, immuno-histological tests, complement-fixation assays, serological tests, immuno-electrophoretic assays, and lateral flow and flow through tests (i.e., rapid “strip” tests). However, there remains a need in the art for improved immunoassays having increased sensitivity. Enzyme-linked immunosorbent assay (ELISA), radioimmunoassay (RIA), chemiluminescence immunoassay (CLIA) are some of the immunoassays with improved sensitivity but are usually equipped with longer processing time and need of expensive equipment and laboratory personnel.

Rapid point-of-care analysis is becoming increasingly important in the diagnosis and treatment of various viral and other pathogenic microbiological agents. Prior art point-of-care tests, such as lateral flow immunochromatography tests, are immunoassays involving an antibody and its antigen.

Lateral flow immunoassays are a subset of antibody/antigen-based immunoassays combining various reagents and process steps in one assay strip, thus providing a sensitive and rapid means for the detection of target molecules. Lateral flow immunoassays are readily applicable for POC testing. Lateral flow immunoassays are available for a wide area of target analytes and can be designed for sandwich or competitive test principles. These lateral flow immunoassays utilize capillary action of multiparous membrane to separate analytes from other constituents. The separation of analytes are dependent upon affinity of the analytes to fixed capture molecules. Membrane based IVDs are cheap, fast, and applicable to a multitude of biological applications.

“Sandwich-type” assays typically involve mixing the test sample with detectable probes, such as dyed latex or a radioisotope, which are conjugated with a specific binding member for the analyte. The conjugated probes form complexes with the analyte. These complexes then reach a zone of immobilized antibodies where binding occurs between the antibodies and the analyte to form ternary “sandwich complexes.” The sandwich complexes are localized at the zone for detection of the analyte. This technique may be used to obtain quantitative or semi-quantitative results.

Lateral flow membranes have pores that are considerably larger than the particles and microspheres used as detector reagents. Overall pore size increases as the flow rate of the membrane increases. The detecting labels used for lateral flow immunoassays (LFAs) have been traditionally gold nanoparticles (GNPs) and, more recently, luminescent nanoparticles, such as quantum dots (QDs). However, these labels have low sensitivity and are costly. Microspheres may flow more slowly than colloidal gold particles through lateral flow membranes and the mobility of microspheres depends on both shape and size.

Generally high molecular weight analytes with several epitopes are typically analyzed in a sandwich format whereas small molecules representing only one epitope are usually detected by means of a competitive assay. Lateral flow assays have a wide array of applications and can test a variety of samples like urine, blood, saliva, sweat, serum, and other fluids. They are currently used by clinical laboratories, hospitals, and physicians for quick and accurate tests for specific target molecules and gene expression. The first tests were made for human chorionic gonadotropin (hCG) for detection of pregnancy.

In the lateral flow test strip (presented in FIG. 2), the liquid sample is applied at one end of the strip, on the adsorbent sample pad that ensures that the analyte present in the sample will be capable of binding to the capture reagents of conjugates and on the membrane depending on their affinity to capture molecules. The treated sample migrates through the conjugate release pad, which contains antibodies that are specific to the target analyte (antigen) and are conjugated to colored or fluorescent particles-most commonly colloidal gold and latex microspheres. The sample, together with the conjugated antibody bound to the target analyte, migrates along the strip into the detection zone.

This is a porous membrane (usually nitrocellulose) with specific biological components (mostly antibodies or antigens) immobilized in lines. Their role is to react with the analyte bound to the conjugated antibody. Recognition of the sample analyte results in an appropriate response on the test line, while a response on the control line (usually detecting gold nanoparticle-antigen-antibody complex) indicates the proper liquid flow through the strip. The read-out, represented by the lines appearing with different intensities, can be assessed by eye or using a dedicated reader.

In developing point-of-care multiple diagnostic assays, a LFA is advantageous in that you can perform it without the use of laboratory investigation, or individuals trained in chemical analysis. LFAs are cheap to produce, easy to use and, importantly, widely accepted by users and regulatory authorities.

However, many conventional lateral flow assays encounter significant inaccuracies when exposed to relatively high analyte concentrations and when attempting to detect very large pathogens that are difficult to cause to flow. When the analyte is present at high concentrations, for example, a substantial portion of the analyte in the test sample may not form complexes with the conjugated probes. Thus, upon reaching the detection zone, the uncomplexed analyte competes with the complexed analyte for binding sites. Because the uncomplexed analyte is not labeled with a probe, it cannot be detected. Consequently, if a significant number of the binding sites become occupied by the uncomplexed analyte, the assay may exhibit a “false negative.” This problem is commonly referred to as the “hook effect.”

The current LFAs based in vitro diagnostic devices using gold nanoparticles all have a much lower limit of detection than other immunological methods or molecular methods. Although the LFAs read the samples containing large amounts of virus as positive, even the most sensitive RAT read the samples containing small amounts of virus, such as early, inactive stage, or asymptomatic stage.

Carboxylate modified latex beads are produced by copolymerizing carboxylic acid containing polymers. The result is a latex polymer particle with a highly charged, relatively hydrophilic and somewhat ‘fluffy’ surface layer. The charge density of the CML particles ranges from about 10 Å2 to 100 Å2 per charged group. They are electrostatically stabilized, and are therefore safe in concentrations of electrolyte up to 1M univalent salt. CML modified latex beads are negatively charged with a surface which has a polyelectrolyte character. It is only when the pH is ˜10 that all the carboxyl groups are ionized.

Severe acute respiratory syndrome coronavirus 2 (SARS-COV-2), which emerged as a novel human pathogen in China at the end of 2019 [1], is responsible for coronavirus disease 2019 (COVID-19), which causes symptoms such as cough and fever, severe pneumonia, and death. The WHO reported that more than 190 million cases of COVID-19, including approximately 4,000,000 deaths, have occurred as of 19 Jul. 2021 (World health h organization, COVID-19 weekly epidemiological update; https://covid19.who.int/). To control the spread of SARS-COV-2 infections, rapid identification and isolation of patients are required.

The laboratory tests for COVID-19 include viral culture, molecular test, and immunologic test. Molecular test includes reverse transcription polymerase chain reaction (RT-PCR) and real time RT-PCR, isothermal nucleic acid amplification, LAMP PCR (digital polymerase chain reaction, microarray analysis, and next-generation sequencing. (Habibzadeh, Parham; Mofatteh, Mohammad; Silawi, Mohammad; Ghavami, Saeid; Faghihi, Mohammad Ali (17 Feb. 2021). “Molecular diagnostic assays for COVID-19: an overview”. Critical Reviews in Clinical Laboratory Sciences: 1-20.)

Reverse transcription-quantitative PCR (RT-qPCR)-based tests is the gold standard to diagnose coronavirus disease 2019 (COVID-19) using nasopharyngeal (N) swabs, throat (T) swabs, or saliva. Reverse transcription-quantitative PCR have to be carried out in laboratories and therefore specimens need to be transported to and examined at sites that have RT-qPCR capability, which delays the test result and increases the anxiety of the suspected COVID-19 patients.

Thus, where RT-qPCR testing capability is lacking, rapid antigen tests (RATs) for COVID-19 based on lateral flow immunoassays are used for rapid diagnosis as it does not require specific and expensive machinery. The most common antigens used for detection of SARS-Cov2 is nucleocapsid protein (N protein, NP) or spike antigen (S protein).

As a result that these tests cannot be done in local clinics where RT-qPCR testing capability is lacking, rapid antigen tests (RATs) for COVID-19 based on lateral flow immunoassays are used for rapid diagnosis. They usually utilize the LFA technology explained above. COVID-19 LFA tests are rapid, cheap, and easy and can be used for self-diagnosis. RATs have been used for mass testing for COVID-19 globally and complement other public health measures for COVID-19.

However, their sensitivity compared with RT-qPCR and other molecular methods range from 11.1-45.7%. Although the RATs read the samples containing large amounts of virus as positive, even the most sensitive RAT read the samples containing small amounts of virus as negative. (Mak, G. C., Cheng, P. K., Lau, S. S., Wong, K. K., Lau, C. S., Lam, E. T., et al. Evaluation of rapid antigen test for detection of SARS-COV-2 virus. J. Clin. Virol. 129, 104500, 2020). This is why it was only considered as an addition to PCR testing. WHO said they “do not currently recommend the use of antigen-detecting rapid diagnostic tests for patient care, although research into their performance and potential diagnostic utility is highly encouraged.” On April 2020 (World Health Organization, Advice on the use of point-of-care immunodiagnostic tests for COVID-19. (https://www.who.int/news-room/commentaries/detail/advice-on-the-use-of-point-of-care-immunodiagnostic-tests-for-covid-19)

However, some rapid tests with increased sensitivity up to 70˜100% and specificity of close to 100% are being reported in Italy, Germany, UK, and Spain. A meta-analysis of the currently available RATs show a sensitivity ranging from 29.5% to 79.8% (average 56.2%), specificity of 98.1% to 99.9%, (average 99.5%). (Dinnes, J., Deeks, J. J., Adriano, A., Berhane, S., Davenport, C., Dittrich, S., et al. Rapid, point-of-care antigen and molecular-based tests for diagnosis of SARS-COV-2 infection. Cochrane Database of Systematic Reviews 2021, Issue 3. Art. No.: CD013705. DOI: 10.1002/14651858.CD013705.pub2.). This means that SARS-COV 2 RATS have value for population-level surveillance and quarantine decisions as it may diagnose half of people with COVID-19 and a positive result is usually a true positive. These RATs may be suitable for the detection of COVID-19 in individuals who are shedding a large amount of SARS-COV-2; that is, they may be useful to identify patients with a high likelihood of transmitting the virus to others. However, the low sensitivity has room for improvement. Commercialized SARS-Cov-2 antigen tests include Panbio COVID-19 Ag Rapid Test (Abbott Rapid Diagnostics) and STANDARD Q COVID-19 Ag Test (SD BIOSENSOR). (Abbott, PANBIO™ COVID-19 Ag RAPID TEST DEVICE. https://www.globalpointofcare.abbott/en/product-details/panbio-covid-19-ag-antigen-test.html; SD BIOSENSOR, STANDARD Q COVID-19 Ag, http://sdbiosensor.com/xe/product/7672). The reason why the current SARS-Cov-2 RATS have limited sensitivity has to do with the fact that conventional LFAs have limited limit of detection for the N protein (nucleoprotein) Antigen is 1 μg/ml˜1 ng/ml.

Thus, what is needed are systems and methods which provided a point of care testing that has improved sensitivity but still has the positive characteristics of LFAs such as rapid, economic and simple-to-use diagnostic tool, since LFA does not require any kind of instrumentation and the result is interpreted at a glance and with very high specificity.

### SUMMARY OF INVENTION

Therefore, according to one aspect of the present invention disclosed herein, there is provided a biosensor device comprising: a lateral flow assay (LFA), wherein the lateral flow assay is a method in a test strip for using a binder conjugated to specific latex bead and a binder conjugated to biotin; and polystreptavidin.

### BRIEF DESCRIPTION OF THE DRAWINGS

FIG. 1 is the Schematic representation of the interaction of Carboxyl Latex Beads (colored latex microsphere)-primary SARS-COV-2 NP mAb-Biotin conjugate capture-SARS-COV-2 Ag-secondary SARS-COV-2 NP mAb-polystreptavidin at the test zone.

FIG. 2 is the schematic representation of the mechanism of lateral flow test strip and interaction of reactants (Carboxyl Latex Beads-Biotin-Polystreptavidin based biosensor and SARS-Cov-2 mAbs, gold nanoparticles-goat anti-chicken antibody) in the course of the assay.

FIG. 3 is a depiction of the configuration of the structure of lateral flow test strip.

FIG. 4 is another depiction of configurations of the structure of lateral flow test strip.

FIG. 5 is a depiction of a gradient score.

FIG. 6 shows quantitative LOD results of the comparison study of COVID antigen test made with gold nanoparticles versus the Carboxyl Latex Beads (1 ng/ml versus 0.1 ng/ml) by LFA reader.

FIG. 7 shows a qualitive results of the comparison study of COVID antigen test made with gold nanoparticles versus the Carboxyl Latex Beads (1 ng/ml versus 0.1 ng/ml).

FIG. 8 shows quantitative results of the comparison study of COVID antigen test made with gold nanoparticles versus the Carboxyl Latex Beads (1 ng/ml versus 0.1 ng/mL) by LFA reader in 30 COVID-19-positive specimens tested by standard RT-PCR testing.

FIG. 9 shows qualitative LOD results of the comparison study of COVID antigen test made with gold nanoparticles versus the Carboxyl Latex Beads (1 ng/ml versus 0.1 ng/mL) upon visual inspection.

FIG. 10 shows qualitative results of the comparison study of COVID antigen test made with gold nanoparticles versus the Carboxyl Latex Beads (1 ng/ml versus 0.1 ng/ml) upon visual inspection in 30 COVID-19-positive specimens tested by standard RT-PCR testing.

FIG. 11 is another illustration of qualitative results of the comparison study of COVID antigen test made with gold nanoparticles versus the Carboxyl Latex Beads (1 ng/ml versus 0.1 ng/ml) upon visual inspection in 30 COVID-19-positive specimens tested by standard RT-PCR testing.

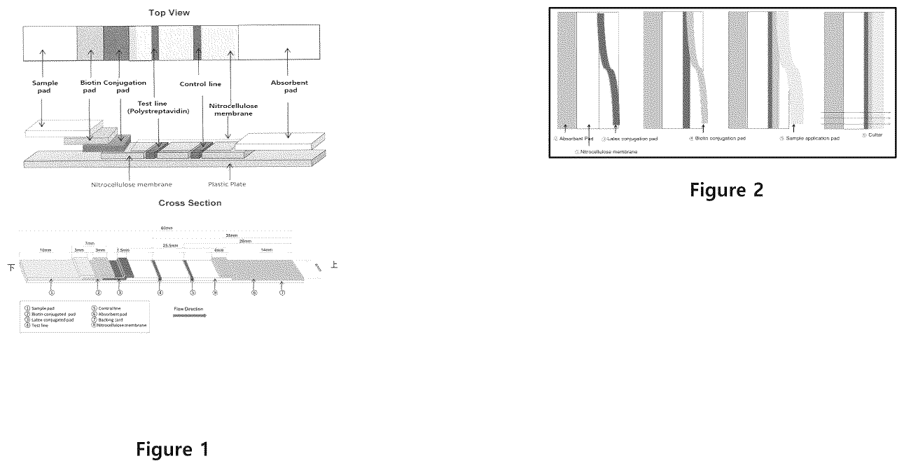

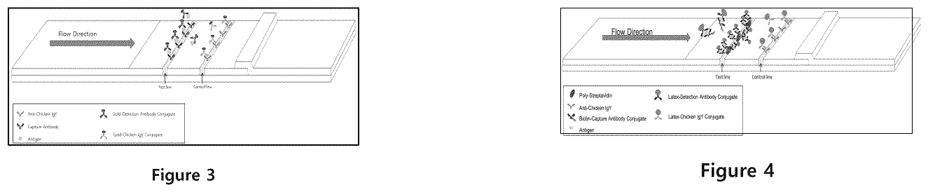

### DETAILED DESCRIPTION

The systems and methods herein are directed to: (i) a point of care of testing and decreasing the limit of detection of N protein Antigen by at least 1/10 of the amount stated above (i.e., 0.1 ng/ml or less); (ii) diagnostic sensitivity which is improved to 90% and above; and (iii) the surveillance and quarantine of the SARS-Cov-2 pandemic. The LFA device for the diagnosis SARS-COV-2 infection and the assay comprise: biotin acting as an immobilization agent, wherein Biotin is bound to primary capture antibodies that can specifically bind the first epitope or the first ligand of the antigen; a Carboxyl Latex Bead is bound to secondary detection antibodies that can bind to the second epitope or second ligand of NP antigen; a test zone; and a control zone. Thus, the NP antigen within the analyte binds to both the primary antibody bound to biotin and secondary antibody bound to Carboxyl Latex Bead. The resulting complex (immune complex of antigen, primary, and secondary antibody) can preferably to be loaded onto the nitrocellulose membrane, where there is a single control line and a single test line. In the test zone, polystreptavidin is at the immobilization site. Functionality for binding the immune complex to the test zone is effected by binding to the biotin in a biotin-polystreptavidin bond, thus amplifying the signal. In the control zone, goat anti-chicken IgY antibodies react with a the chicken antibody present in the latex beads regardless of the presence of target NP antigen.

Accurate rapid diagnostic tests for SARS-COV-2 infection could contribute to clinical and public health strategies to manage the COVID-19 pandemic. Point-of-care antigen and molecular tests to detect current infection could increase access to testing and early confirmation of cases, and expediate clinical and public health management decisions that may reduce transmission.

Point-of-care antigen tests that were previously developed usually are LFA tests that utilize gold nanoparticles, and the systems and methods herein aims to overcome technical difficulties related to previous antigen tests, develop a cheap, cost-effective, easy POC LFA antigen test with better limit of detection and sensitivity.

The systems and methods herein are related to lateral flow devices that utilize optical detection mechanism to quantify analyte with improved sensitivity and high precision for immunoassay for COVID-19. Device and methods thereof are disclosed for easily and rapidly analyzing COVID-19 samples utilizing Carboxyl Latex Beads as a label agent to increase the emission intensity for sensitive COVID-19 N antigen detection in membrane chromatography.

The systems and methods herein increase the immune response between the target antigen and antibody. For this, Carboxyl Latex Beads were chosen in place of gold nanoparticles for in vitro diagnosis and the surface of these Carboxyl Latex Beads was modified with EDC/NHS. The resulting reactive macromolecule and nonreactive macromolecule are affixed to the surface of the latex microspheres, and inside, the dye is stably fixed by covalent bond, and this was conjugated to the secondary detection antibody.

The two methods herein include: (1) the strength of immune reaction by using Carboxyl Latex Beads and (2) signal amplification using Biotin-polystreptavidin, which could increase the limit of detection of the LFA method compared to conventional gold nanoparticles by 2 to 20 times. This has the advantage of being stable from heat, pH changes, and other environmental changes, while being stable even with prolonged preservation. Multiple dyes may be trapped inside the latex microsphere in the LFA-based immunochromatography and the resulting color in the latex microsphere may be amplified for signal because the light absorption is amplified upon detection of reaction. Each molecule within the multiple dyes as a combination absorbs light more than per dye molecule and this enables limit of detection, which is often a problem in LFA assays, to be overcome.

Second, a method for amplifying the signal from immune reaction is disclosed. For signal amplication, biotin-polystreptavidin/streptavidin method is used, wherein Biotin is conjugated to the Fc portion of the primary capture antibody and polystreptavidin is affixed to the test line resulting in amplified signal. The polystreptavidin is polymer made from streptavidin bound to dextran and a plurality of streptavidin particles (i.e., more than one) are able to get into the space within polymer frame. This enables: (i) a single polystreptavidin to bind to multiple biotin and (ii) multiple detection marker reactions, thereby resulting in an amplified signal and improved detection limit. Streptavidin can bind very closely to Biotin molecule and this streptavidin coating on solid surface offers a substrate in which to attach proteins, peptide, PCR fragments, haptens, etc. Polystreptavidin coating can exhibit improved binding properties, high chemical/heat stability, high absorption properties and less propensity for nonspecific binding. This is very applicable to coating of membranes, beads, bio-chips, plastic, etc. Polystreptavidin comprises 12 biotin attachment sites per macromolecule and can exhibit such strong binding ability and amplification.

The systems and methods herein also include a kit for rapid diagnosis of NP antigen for COVID-19 (rapid COVID-19 antigen test kit, Cellgenemedix). Clinical performance evaluation using human nasopharyngeal swab specimens for COVID-19 showed sensitivity 95% and 100% specificity compared to Real-time RT-PCR, which is considered the gold standard in the art. This is superior to all the Rapid antigen test kits in commercial use. The kit of the systems and methods herein is applicable to many clinical situations as diagnosis is made by visual inspection and results come out with no other additional devices. In addition, the results come out within 20 minutes and this makes it very appropriate for point of care testing and self-diagnosis. Mass production is possible and price of production is cheap.

Advantage 1: The systems and methods herein produce an easy, cost-effective method to diagnose SARS-COV-2 utilizing Carboxyl Latex Beads and biotin-polystreptavidin instead of gold nanoparticles to overcome the previous limitation of low limit of detection and sensitivity.

Advantage 2: The systems and methods herein disclose 0.1 mg/ml of limit of detection and is expected to be valuable in that it is cost-effective, easy to use and can aid in multiple applications such as, but not limited to, rapid, early diagnosis and prevention of spread, quarantine, epidemiological studies, prognosis, and treatment response.

Example 1—Production of Carboxyl Latex Bead and Biotin biotin-polystreptavidin lateral flow assay, (CL-B/PS LFA) The systems and methods herein are directed to a Carboxyl Latex Bead and Biotin-streptavidin utilizing LFA (CL-B/PS LFA).

### Manufacturing Details

The ingredients for CL-B/PS LFA include the following below.

The Carboxyl Latex Bead was purchased from Magspher Inc (CA, USA); 400 nm-sized Carboxylic red latex beads is 400 nm (CAB400NM, CAB4865B-1220).

The NHS-biotin (20217, VJ309468) was purchased from Thermo Scientific (MA, USA).

The latex Sulfo-NHS (24510, VL308261), as used in this example, provided microsphere conjugation. The latex Suflo-NHS was purchased from Thermo Scientific InC (MA, USA) and EDC (E7750, BCCD7592) from Sigma-Aldrich InC (St. Louis, USA).

The polystereptavidin, as used in this example, was Polystreptavidin R (ABIN4370319, K664-M/250321) and purchased from Antibodies InC (Aschen, Germany).

The standard antigen for SARS-COV-2 included: a nuclear protein (NP) antigen made by recombinant DNA technology from E. coli using recombination technology; and purified (COVID-19 NP Rec. Ag. BHAG-N3, M210303). This has been purchased from Boreda biotech, InC (Seong nam, South Korea), but not restricted to this.

For primary and secondary antibodies, 2 monoclonal antibodies made for NP antigen of Sars-COV; SARS-COV-2 NP mAb (#67012, CB25172); and SARS-COV-2 NP mAb (#67011, CB24857) was used and purchased from Boreda biotech, InC (Seong nam, South Korea), but not restricted to this.

The Goat anti-chicken IgY antibodies (20900, CB25203) was used for control line and purchased from Boreda biotech, InC (Seong nam, South Korea), but not restricted to this.

Boric acid (B0394), Casein (C7078), MES hydrate (M2933), EDC (3003026795), Sodium phosphate monobasic (S0751), Sodium phosphate dibasic (S0876), Tween-20 (P2287), BSA (A7906) was purchased from Sigma-Aldrich InC (St. Louis, USA).

The striping of the systems and methods herein included: sample pad, conjugation pad, nitrocellulose membrane), and absorbent pad, with backing card. The nitrocellulose membrane (IAB090-E1, 00221107) was purchased from Advantech InC (Tai-pei, Taiwan), and absorbent pad (Grade 222, 113903), sample pad (Grade 319, 113863) and conjugation pad (Grade 8964, 003171) was purchased from AHLSTROM Co. Ltd., (Helsinki, Finland). Backing card was purchased from PJGo, InC (Seoul, Republic of Korea).

### Carboxyl Latex Bead and Bound Detection Antibody Production

The systems and methods herein increased the affinity of target antigen and antibody. More specifically, Carboxyl Latex Bead instead of gold nanoparticles were chosen and Carboxyl modified latex bead was surface-treated with EDC/NHS for in vitro diagnostic use. The resulting reactive and nonreactive macromolecule made with dye fixed in a covalent bond was fixed on the surface and antibody. The resulting molecule was resistant to heat and pH changes and remained stable and can be preserved for longer periods of time.

Carboxylated PS Latex Particles, as purchased from the Magspher, InC (Pasadena, CA 91107 U.S.A.), was agitated to prevent agglutination and ultrasonic waves are applied according to manufacturer's instructions. (https://www.magsphere.com/Products/Carboxylated-Latex-Particles/carboxylated-latex-particles.html). Microspheres were monodisperse before starting the protocol before prior to no aggregation, as confirmed by microscope.
1. The Carboxylated PS Latex particles were put into 0.1 M MES (PH 6.1) buffer. Ultrasonic waves were applied for uniformization. Centrifugation at 1 2,000 rpm for 20 minutes are applied, after which supernatant was removed a nd washed. This was resuspended in 0.1 M MES (PH 6.1) buffer.
2. These beads were attached by covalent bond to antibodies in a 2-step process: MES buffer was used to activate the beads by mixing with EDC an d Sulfo-NHS. Thirty minutes later, beads were washed and resuspended in MES buffer.
3. 4 mg/ml concentration of #66104 monoclonal antibodies were attached to beads using EDC/NHS coupling in the mixer for 3 hours, centrifuged, an d supernatant was removed.
4. The resulting beads were mixed for 30 minutes in a 0.2% ethanolamin e solution.
5. For the control line: 400 nm Carboxylate modified latex bead was applied at a 4 mg/ml concentration Chicken IgY (Boreda Biotech, Seoul, South Korea) using EDC/NHS coupling. This was mixed in 0.2% ethanolamine solution for 30 minutes.
6. 10% casein was added to a final concentration of 1.5%.
7. The microspheres were put into mixer in room temperature for 1 hour. After 1 hour, the content was centrifuged and the supernatant was removed.
8. Resulting microsphere was conserved in 1% casein 100 mM borate buffer.
9. The concentration of resulting microspheres was chosen after measuring the light absorptance at 660 nm. Final microspheres were preserved at 4° C.

### Manufacturing of Biotin and Secondary Detect Antibody Complex

The second point of the systems and methods herein was to increase the signal generated where Biotin and streptavidin, especially Polystreptavidin, were used. The Fc portion of primary capture antibody was affixed to Biotin and test zone (test line) had Polystreptavidin, such that the resulting signal was amplified when the biotin-primary capture antibody complex reacted with polystreptavidin. Streptavidin had a high affinity to Biotin, especially solid phase streptavidin coating allowed for receiving: protein, peptide, PCR fragments, nucleic acid, hapens, and so forth. The polystreptavidin of the systems and methods herein had an even higher affinity for biotin than streptavidin. Polystreptavidin had 12 Biotin binding sites per molecule which allows for stronger bond and amplified signal. The coating by polystreptavidin had strong affinity, high stability to chemical and heat, high absorption capacity, and low nonspecific binding, thus making it appropriate for coating membranes, beads, biochips, and plastic, etc.

### Biotin-Primary Capture Antibody Manufacture

SARS-COV-2 NP mAb (#67011, CB24857) monoclonal antibody was diluted in 50 mM Sodium Phosphate buffer (PH7.5) into 1 mg/ml. NHS-Biotin (20217, Thermo Scientific) was added into monoclonal antibodies offered at 1 mg/mL into a concentration of 10 mM. The final concentration of the biotinized antibodies were measured by A280. Final mole concentration of Biotination was 20 biotin per mole of antibody using the QuantTag Biotin kit (BDK-2000, Vector Laboratories, UK).

### Manufacture of LFA Strip for Device

The strip of the systems and methods herein constituted of sample pad, conjugation pad, nitrocellulose membrane, absorbent pad, and backing card. The uncut sheet was inserted to the cutter to be cut in sizes of 60 mm×4 mm. The cut strip was put together into the cassette as below in the correct direction, as described below.

Backing Card (PJGo InC, Seoul, Korea) was attached into nitrocellulose membrane (Nupore Filtration Systems Pvt. Ltd.). Polystreptavidin 1.5 mg/mL solution was prepared in 50 mM SPB and Goat anti-chicken IgY antibody 1.5 mg/mL solution in 50 mM PBS using the Dispenser system (Zeta Co., Seoul, Korea). The test line was 35 mm above the upper part of nitrocellulose membrane and the control line was 26 mm above the upper part of the nitrocellulose membrane at a speed of 0.8 ul/cm. The cut strip was dried at 37° C. temperature for 3 hours. The absorbent pad (AHLSTROM) was attached so that there was an overlap between the membrane 4 mm on the upper part of backing card. The secondary detection antibody (#66105) was: conjugated into Carboxyl Latex Beads and applied to the conjugation pad at a concentration of 3.4%. The control line antibody complex was applied at the control zone at a concentration of 1.3%. The dried conjugation pad was cut to a size of 300 mm×7 mm after which, the pad was attached to the strip, such that overlap with the membrane was 1.5 mm on the upper part of nitrocellulose membrane. The primary Capture antibody after conjugation with biotin was applied at a concentration of 0.34% and let dry; after cutting into a size of 300 mm×7 mm and attaching, such that there was 3 mm overlap with the latex pad. Finally, the sample pad was cut to a size of 300 mm×13 mm attached, such that there was 3 mm overlap with biotin conjugation pad to finished uncut sheet, as depicted in FIG. 2. The manufacturing process of lateral flow chromatography is further described in FIG. 2.

### Example 2-Manufacturing of Immunochromatography Using Gold Nanoparticle

LFA immunochromatography assay of the systems and methods herein was used as a comparison method using gold nanoparticles instead of Carboxyl Latex Beads and Biotin-Polystreptavidin.

### Ingredients

Through Boreda biotech, InC (Seong nam, South Korea), the COVID-19 NP Rec. Ag (BHAG-N3, M210303), primary, secondary monoclonal antibodies (SARS-COV-2 NP mAb (#67012, CB25172) and SARS-COV-2 NP mAb (#67011, CB24857)), Goat anti-chicken IgY antibodies (20900, CB25203) for the control line, and gold nanoparticles (#BBCG-40, G210602JM-40) were used to make the comparison method.

### Manufacturing Details

The ingredients for the example of comparison LFA method using gold nanoparticles was purchased as below.

The main difference was the use of 40 nm gold nanoparticles (#BBCG-40, G210602JM-40) instead of the Carboxyl Latex Bead (Magspher InC (CA, USA); 400 nm-sized Carboxylic red latex beads (400 nm, CAB400NM, CAB4865B-1220)). Also, the polystereptavidin and NHS-biotin was not used and secondary antibodies were instead directly applied to the test zone.
1) 1 mL of gold nanoparticle was diluted into 1× concentration (1.40×10¹²/mL) in a 1.5 mL EP tube by using 1 mL 0.1 M borate buffer (pH 8.5), after which 30 seconds of vortex mixing and spin down of 10 seconds is applied. This was set in 25° C. for 10 minutes.
2) 1 mg/ml of Primary capture antibodies (COVID-19 mAb, #67011) were added; vortexed for 30 seconds and centrifuged (25 rpm); and set in 25° C. for 1 hour.
3) 1 mL of 1× phosphate buffered saline (PBS, pH 7.4) was added 20% (g/g) BSA; vortexed for 10 seconds and centrifuged (25 rpm); and set in 25° C. for 30 min (blocking process).
4) Washing was done as follows for the 40 nm gold nanoparticles: centrifuge (12,000 rpm, 4° C., 25 min) after which supernatant is thrown away and 1 mL of 10 mM borate buffer (pH 8.5) added and set so that the pellet in the lower part of the EP tube is loosened, and the same centrifuge process for 3 times (×3).
5) Backing Card (PJGo InC, Seoul, Korea) was attached into a nitrocellulose membrane (Nupore Filtration Systems Pvt. Ltd. Secondary detection antibodies (COVID-19 mAb, #67012) were prepared as a 1.5 mg/mL solution in PBS and Goat anti-chicken IgY antibody as a 1.5 mg/mL solution in 50 mM SPB using the Dispenser system (Zeta Co., Seoul, Korea) at a speed of 0.8 ul/cm on the nitrocellulose membrane for the test and control line. This was dried in 37° C. for 3 hours.
6) The strip in the systems and methods herein constituted: a sample pad, a conjugation pad, a nitrocellulose membrane, and an absorbent pad with backing card. The uncut sheet was inserted to the cutter to be cut in sizes of 60 mm×4 mm. The cut strip was put together into the cassette as below in the correct direction as below.

### Example 3—Strip Analysis of New Lateral Flow Immunoassay Using Carboxyl Latex Beads and Biotin-Polystreptavidin

An optimal method for the interpretation of the new immunochromatography method used the Latex Beads and Biotin-Polystreptavidin as in Example 1 for the detection of NP antigen of SARS-Cov-2 virus with high sensitivity and efficacy. This was used to show that the biosensor device in Example 1 was more efficient than the comparison biosensor method using gold nanoparticles in Example 2.

Eye Test: A first option was to read the assay by eye. This was acceptable for positive/negative scoring but is not useful for quantitative assays. The systems and methods herein included: gradient score cards where the strength of the lateral flow line that can be measured against a printed line intensity in order give a semi-quantitative score.

Scanner: Alternatively, dedicated commercial reader was captured in an image of the test line. The color density (and thus line strength) was analyzed with an image processing program, resulting in a number that directly correlates to the test line intensity. Readers from companies, such as Lumos Diagnostics and Qiagen, was used that lead to a quantitative readout in as little as 30 seconds.

With respect to the scoring card and interpretation of results, multiple concentration levels of SARS-Cov-2 virus NP antigen was prepared by: mixing in buffer and apply it to the application zone (sample well) in the LFA device. Afterwards, the test line and control line was inspected after 20 minutes to interpret the results.

For positive results, there were two distinct colored lines that appear (e.g., one red-colored line next to “C” and one red-colored line next to “T” indicate COVID-19 positive result). The color intensity in the test region varied depending on the amount of SARS-COV-2 nucleocapsid protein antigen present in the sample. Any faint colored line(s) in the test region(s) should be considered as positive.

For negative results, there was one red-colored line only next to “C” and there was no line (below 2/10) in the “T” zone.

For invalid results, there was the red-colored line in the control region “C” that was not visible. The test was re-run one time using the remaining specimen in the extraction vial if an invalid result was obtained during initial testing and resets using other methods should be considered.

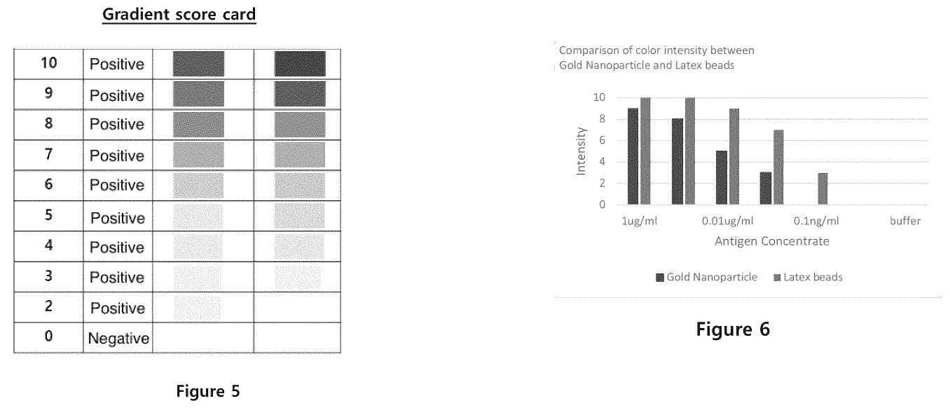

With respect to the scoring card, The signal intensity of latex beads and gold nanoparticles were scored from 0 to 10. The minimum visible amount of signal detectable by the eye was considered a “2”, the maximum “10”, and any color/detectible signal between 2-10 was considered positive for the line. (See FIG. 5.) In systems and methods herein, the latex-biotin-polystreptavidin based biosensor in the Example 1 showed a red signal and the gold based biosensor in Example 2 showed a red signal. Semiquantification of antigen (antigen-antibody complex) was done by LFA reader but the direct quantification of the signal has not been proven to date as this depends on multiple variables.

The amount of antigen in the test was proportional to the amount of N antigen in the analyte. However, due to the reasons stated above the main purpose of the systems and methods herein was qualitative detection of SARS-Cov-2 infection.

10 ul of analyte (SARS-Cov-2 NP antigen/positive control) was mixed with extraction buffer 90 ul and applied (100 ul) to the application zone in the LFA device after which the reaction was carried out for 20 minutes. The test line and control line were inspected after 20 minutes to interpret the results.

The gradient score cards where the strength of the lateral flow line were measured against a printed line intensity in order give a semi-quantitative scores, as depicted in FIG. 6 (0˜10/red for gold/red for latex bead).

### Example 4—Analytical Performance Evaluation of the Good Ag COVID-19 Antigen Test Utilizing Latex Beads and Biotin-Polystreptavidin

### [Example 4-1A] Limit of Detection (LoD) in Comparison with Antigen Test Utilizing Gold Nanoparticles

### Material and Methods

The LoD for direct swab was established using SARS-COV-2 positive control nucleoprotein
antigen made by E. coli recombination technology (COVID-19 NP Rec. Ag. BHAG-N3,
M210303, Boreda Biotech, Seoul, South Korea).

Multiple concentration levels of control material (10 uL, COVID-19 NP Rec. Ag. BHAG-N3,
M210303) was mixed into 90 ul of extraction buffer and applied to the application area (total
of 100 uL) after which the test result was read and interpreted at 20 minutes and compared
with the score card by eye exam. The initial concentration of SARS-CoV-2 positive control
nucleoprotein antigen in the sample applied to the sample well was as follows: 1 μg/ml, 0.1
ug/ml, 0.01 ug/ml, 1 ng/ml, 0.1 ng/ml, 0.01 ng/ml, and 0 ng/ml. The initial serial dilution
test was tested in 20 replicates. Scoring of the antigen test was done by eye exam as in
Example 3 by comparing the color of the test card with the provided score card and results
were shown.

The estimated LoD found from the initial serial dilution test was confirmed by testing 20
replicates again in serial concentration levels as follows: 10 ng/ml, 1 ng/ml, 0.5 ng/ml, 0.1
ng/ml, 0.05 ng/ml, 0.01 ng/ml, and 0 ng/ml. LoD is defined as the lowest amount of a target
or analyte that the assay can detect positive signal 95% of the time.

### Result

The analytical sensitivity and signal intensity of kit was made using Gold nanoparticles. A
comparison with the kit using Gold nanoparticles was made with a kit made using Carboxyl
Latex Beads and Biotin-Polystreptavidin for Covid-19 diagnosis, as shown in Tables 1 and 2.

Carboxyl Latex Beads and Biotin-Polystreptavidin using kit (Example 1) showed: superior
clinical performance compared to gold nanoparticle kit (Example 2) and higher signal
intensity in all dilution levels.

### Results

The analytical sensitivity and signal intensity of kit were made using Gold nanoparticles. A
Comparison with the kit using Gold nanoparticles was made using Carboxyl Latex Beads and
Biotin-Polystreptavidin for Covid-19 diagnosis, as depicted in FIGS. 4 and 5.

The color signal at Carboxyl Latex Beads Biotin-Polystreptavidin immunochromatography
versus color signal at gold nanoparticles was shown 10 times as high as the signal and
sensitivity in the Carboxyl Latex Beads Biotin-Polystreptavidin method.

TABLE 2
analytical sensitivity of kit made using Carboxyl Latex Beads
Biotin-Polystreptavidin method for Covid-19 diagnosis

| | | | | PPA |
|---|---|---|---|---|
| | | | | (posititive |
| | | Number | | percent |
| Specimen | Specimen | of | Positive | agreement) |
| No. | Concentration | replicates | Replicates | (%) |
| 1 | 1.00 ug/ml | 20 | 20 | 100 |
| 2 | 0.10 ug/ml | 20 | 20 | 100 |
| 3 | 0.01 ug/ml | 20 | 20 | 100 |
| 4 | 1.00 ng/ml | 20 | 20 | 100 |
| 5 | 0.10 ng/ml | 20 | 20 | 100 |
| 6 | 0.01 ng/ml | 20 | 10 | 50 |
| 7 | 0.00 ng/ml (buffer) | 20 | 0 | 0 |

TABLE 2
analytical sensitivity of kit made using Gold nanoparticles for
Covid-19 diagnosis

| | | | | PPA |
|---|---|---|---|---|
| | | | | (posititive |
| | | Number | | percent |
| Specimen | Specimen | of | Positive | agreement) |
| No | concentration | replicates | Replicates | (%) |
| 1 | 1.00 ug/ml | 20 | 20 | 100 |
| 2 | 0.10 ug/ml | 20 | 20 | 100 |
| 3 | 0.01 ug/ml | 20 | 20 | 100 |
| 4 | 1.00 | 20 | 20 | 100 |
| 5 | 0.10 ng/ml | 20 | 8 | 40 |
| 6 | 0.01 ng/ml | 20 | 0 | 0 |
| 7 | 0.00 ng/ml (buffer) | 20 | 0 | 0 |

The color signal at current invention versus color signal at method using gold nanoparticles was shown 10 times as high as the signal and sensitivity in the current invention. The confirmed LoD for direct swab was 0.1 ng/ml to 0.01 ng/ml using Good Ag test (Example 1) with 100% detection rate at 0.1 ng/ml LOD. However, the LOD for gold nanoparticle (Example 2) ranged from 1 ng/ml to 0.1 ng/ml and only 40% detection rate at 0.1 ng/ml, as shown in Tables 1 and 2. (FIG. 6)

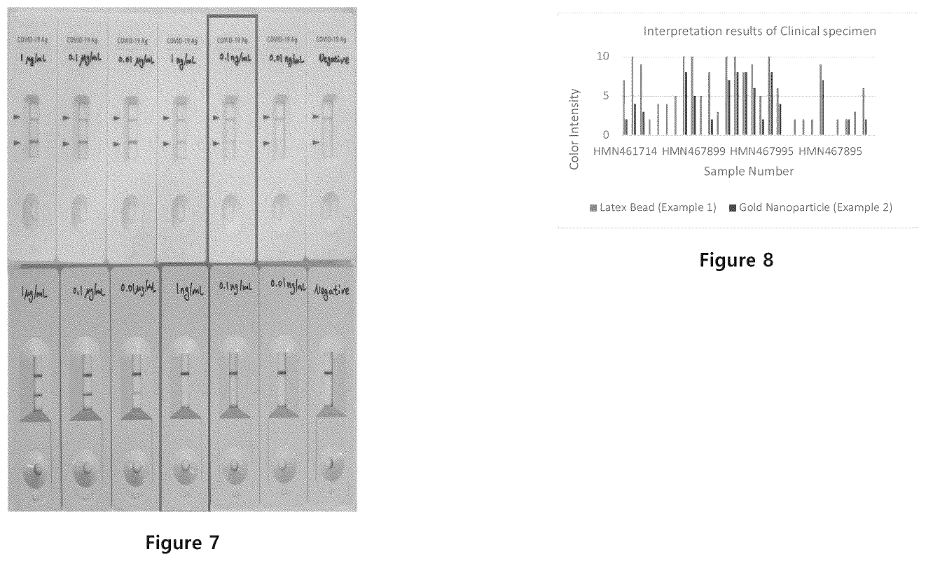

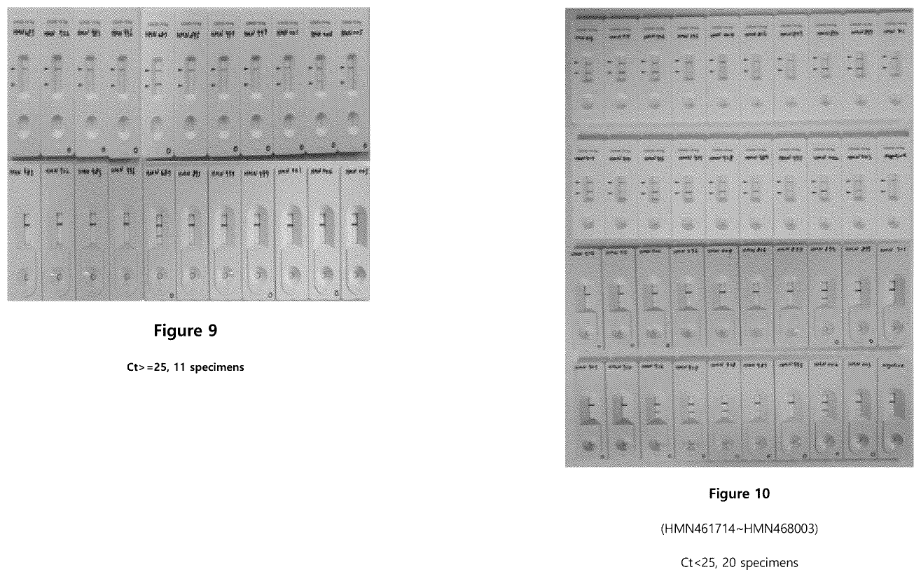

The confirmed LoD for direct swab was 0.1 ng/ml to 0.01 ng/ml using Carboxyl Latex Beads Biotin-Polystreptavidin immunochromatography (Example 1) with 100% detection rate at 0.1 ng/ml LOD. However, the LOD for gold nanoparticle (Example 2) ranged from 1 ng/ml to 0.1 ng/ml and only 40% detection rate at 0.1 ng/ml, as shown in Tables 1 and 2 and FIGS. 6 and 7.

To summarize, the assay using CL-B/PS LFA detected NP antigen of SARS Cov-2 95% of the time at 0.1 ng/mL versus assay using gold nanoparticles in 1 ng/ml. Thus, LOD (lowest amount of a target or analyte that the assay can detect positive signal 95% of the time) was 10 times more in the comparison method, proving the superior performance of the assay using Carboxyl Latex Beads Biotin-Polystreptavidin immunochromatography versus assay using gold nanoparticle immunochromatography. (FIG. 7; at this time, Good ag was carried out with blue beads for comparison)

### [Example 4-1B] Limit of Detection (LoD)

A preliminary LoD was determined by evaluating different concentrations of a SARS-COV-2 dead-attenuated virus stock (SARS-CoV-2, heat-inactivated, ATCC VR-1986 HK) diluted natural nasal clinical matrix. Contrived nasal swab samples were prepared by absorbing 100 microliters of each virus dilution onto the swab. The contrived swab samples were tested according to the test procedure. Contrived samples were randomized, and operators were blinded to the sample identities for testing on the Good Ag COVID-19 Antigen Test. The LoD was confirmed as the lowest concentration of SARS-CoV-2 that was detected ≥95% of the time (i.e., concentration where 19 out of 20 test results were positive). The Good Ag COVID-19 Antigen Test LoD was confirmed to be 6.5×10\hat{ }¹TCID50/mL (Ct: 27.35).

### [Example 4-2] Stability

The specimen stability of a nasopharyngeal swab specimen with the Good Ag COVID-19 Antigen Test was evaluated with SARS-COV-2 negative and spiked positive specimens at 2×LoD. The stability at room temperature was evaluated by placing the samples in a dry tube and stored at 30° C., for up to 180 minutes; the stability at refrigerated temperature was evaluated by storing the samples at ˜4° C., for up to 36 hours. No false results were obtained during the study. The optimal storage requirement of the GG COVID-19 Real-Time PCR Kit was set at 12 months (32.3 days once opened). Accelerated aging time calculated results at above 30 days, corresponding to 12 months showed as results below. Avoiding of excessive freeze/thaw cycles for reagents is recommended.

Corona-19 virus (SARS-COV-2, heat-inactivated, ATCC VR-1986 HK) is made into a sample of 13×10\hat{ }1 TCID_{SO}/ml and then added to the negative sample to make a positive sample. Viruses of each concentration were put in an injection buffer tube containing 400 UL of extract to extract the virus, and 100 μL of this was instilled at the sample instillation site, and the results were read 20 minutes later. Kit preparation: 3 different of Lot Response: Dependent on dosage. Sample preparation: prepare as 13×10¹-TCID₅₀/ml, negative Drop 100 sheets of diluted sample extract on the sample dripping site. The reading time is 20 minutes, and if it can be judged as positive, it can be judged from 20 minutes, and the result after 30 minutes is not read.

Based on the safety test standards for medical devices, ([Enforcement 2020. 5. 1.] [Ministry of Food and Drug Safety Notice No. 2020-29, the accelerated aging time was calculated and tested according to the table below
AAF=accelerated aging factor
Q₁₀=conservative aging factor (normally Q10 is 2.0)
TAA=accelerated aging temperature in ° C. (temperature aging study is conducted)
TRT=ambient temperature in (storage temperature for real-time aging, normally 25° C.)
RT=required shelf life
AAF=Q₁₀\hat{ }^{[(TAA-TRT)/10]}
AAT=RT/AAF
   1) 60° C.

| Aging coefficient | Q10 | 2 |
|---|---|---|
| Accelerated aging | TAA | 60 |
| temperature (° C.) | | |
| Storage temperature (° C.) | TRT | 25 |
| Accelerated aging | AAF = | 11.3137085 |
| coefficient | Q10[(TAA-TRT)/10] | |
| | Period (days)/accelerated | Accelerated aging |
| | aging coefficient | time (days) |
| Valid for 3 | 90/11.3137085 | 8.0 |
| months | | |
| Valid for 4 | 120/11.3137085 | 10.6 |
| months | | |
| Valid for 6 | 180/11.3137085 | 15.9 |
| months | | |
| Valid for 12 | 365/11.3137085 | 32.3 |
| months | | |

Kit preparation: 3 lot kits 3 times each (0, 3, 4, 6, 12 months) for a total of 15 times.

| | Period (days)/ | Accelerated |
|---|---|---|
| | accelerated | aging |
| | aging coefficient | time (days) |
| Valid for 3 | 90/5.6568542 | 15.9 |
| months | | |
| Valid for 4 | 120/11.3137085 | 212 |
| months | | |
| Valid for 6 | 180/113137085 | 31.8 |
| months | | |
| Valid for 12 | 365/11.3137085 | 64.5 |
| months | | |
| Aging coefficient | Q10 | 2 |
| Accelerated aging | TAA | 50 |
| temperature (° C.) | | |
| Storage temperature (° C.) | TRT | 25 |
| Accelerated aging coefficient | AAF = | 5.6568542 |
| | Q10[(TAA-TRT )/10)] | |

Kit preparation: 3 lot kits 3 times each (0, 3, 4, 6, 12 months) for a total of 9 times.

### Criteria:

The result of the test with the Corona-19 virus (SARS-COV-2, heat inactivated, ATCC VR-1986HK) is checked with the reading reference color chart and the Ct value obtained by Real-time PCR, and then the result is judged.

1. 60° C. Experiment result

| Storage | Storage | | | |
|---|---|---|---|---|
| temperature | period | Lot 1 | Lot 2 | Lot 3 |
| 60 ± 2° C. | 0 day | 9/9 | 9/9 | 9/9 |
| | | (Positive) | (Positive) | (Positive) |
| | 8 days | 9/9 | 9/9 | 9/9 |
| | | (Positive) | (Positive) | (Positive) |
| | 11 days | 9/9 | 9/9 | 9/9 |
| | | (Positive) | (Positive) | (Positive) |
| | 16 days | 9/9 | 9/9 | 9/9 |
| | | (Positive) | (Positive) | (Positive) |
| | 32 days | 9/9 | 9/9 | 9/9 |
| | | (Positive) | (Positive) | (Positive) |

2. Experiment result in 50° C.

| Storage | Storage | | | |
|---|---|---|---|---|
| temperature | period | Lot 1 | Lot 2 | Lot 3 |
| 50 + 2° C. | 0 day | 9/9 | 9/9 | 9/9 |
| | | (Positive) | (Positive) | (Positive) |
| | 16 days | 9/9 | 9/9 | 9/9 |
| | | (Positive) | (Positive) | (Positive) |
| | 21 days | 9/9 | 9/9 | 9/9 |
| | | (Positive) | (Positive) | (Positive) |
| | 32 days | 9/9 | 9/9 | 9/9 |
| | | (Positive) | (Positive) | (Positive) |
| | 65 days | 9/9 | 9/9 | 9/9 |
| | | (Positive) | (Positive) | (Positive) |

### [Example 4-5] Cross-Reactivity (Analytical Specificity) and Microbial Interference

Cross-Reactivity and Microbial Interference studies were conducted to determine if other respiratory pathogens that could be present in a nasal sample could cause a false-positive test result, or interfere with a true positive result. No cross-reactivity or interference was seen with the following microorganisms when tested at the concentrations listed in the table below with the exception of SARS-COV, which resulted in positive test results due to the high homology between SARS-COV and SARS-COV-2 nucleocapsid proteins.

| no | Cross Reactant | Concentration |
|---|---|---|
| 1 | Recombinant SARS-CoV | 3 ug/ml |
| 2 | Human Coronavirus NL63 | 1.4 × 10⁵ |
| | | TCID₅₀/ml |
| 3 | Human Coronavirus 229E | 1.2 × 10⁵ |
| | | TCID₅₀/ml |
| 4 | Human Coronavirus OC43 | 0.9 × 10⁵ |
| | | TCID₅₀/ml |
| 5 | Influenza A H1N1 | 2.5 × 10⁵ |
| | | TCID₅₀/ml |
| 6 | Influenza A H3N2 | 2.9 × 10⁵ |
| | | TCID₅₀/ml |
| 7 | Influenza B | 2.2 × 10⁵ |
| | | TCID₅₀/ml |
| 8 | syncytial virus Type A | 1.5 × 10⁵ |
| | | TCID₅₀/ml |
| 9 | Parainfluenza virus Type 1 | 1 × 10⁶ |
| | | TCID₅₀/ml |
| 10 | Parainfluenza virus Type 2 | 0.9 × 10⁶ pfu/ml |
| 11 | Parainfluenza virus Type 3 | 9.5 × 10⁶ pfu/ml |
| 12 | Rhinovirus A30 | 4.4 × 10⁵ pfu/ml |
| 13 | Adenovirus type 1 | 3.6 × 10⁶ pfu/ml |
| 14 | Adenovirus type 3 | 2.5 × 10⁵ pfu/ml |
| 15 | Adenovirus type 5 | 0.8 × 10⁷ pfu/ml |
| 16 | Adenovirus type 6 | 4.4 × 10⁵ pfu/ml |
| 17 | Adenovirus type 8 | 1.8 × 10⁶ pfu/ml |
| 18 | Adenovirus type 11 | 4.0 × 10⁵ pfu/ml |
| 19 | Adenovirus type 19 | 2.4 × 10⁷ pfu/ml |
| 20 | Streptococcus pneumonia | 1 × 10⁵ cfu/ml |
| 21 | Legionella pneumophila | 1 × 10⁶ cfu/ml |
| 22 | Candida albicans | 1 × 10⁶ cfu/ml |
| 23 | Pseudomonas aeruginosa | 1 × 10⁶ cfu/ml |
| 24 | Staphylococcus | 1 × 10⁶ cfu/ml |
| | epidermidis | |
| 25 | Streptococcus salivarius | 1 × 10⁶ cfu/ml |
| 26 | Haemophilus influtenzae | 1 × 10⁶ cfu/ml |

Additionally, the SARS-COV-2 Nucleocapsid protein sequence was BLAST aligned on the NIH NCBI database to the entire set of proteins encoded by P. jirovecii. No significant identity was found as a result of this search and thus no interference is expected with the Good Ag COVID-19 Antigen Test, however, cross-reactivity cannot be ruled out.

### [Example 4-4] High Dose Hook Effect

Potential hook effect in the Good Ag COVID-19 Antigen Test was assessed by loading 100 μL of neat virus stock directly onto the center of the flat pad of test device in triplicate, resulting in a test concentration of 5.0×10\hat{ }5 TCID50/mL. No hook effect was seen with the USA-WA1/2020 SARS-COV-2 isolate.

### [Example 4-3] Endogenous Interfering Substances

A study was conducted to determine if any substances, naturally present in respiratory specimens or that may be artificially introduced into the nasal cavity listed in the table interfere in the performance of the Good Ag COVID-19 Antigen Test. In addition to the materials that are found in the nasal cavity, substances that are commonly found on the hands were also tested. Test performance was evaluated in the absence and presences of SARS-COV-2 (3×LoD). None of the substances listed in the tables below interfered with the performance of the Good Ag COVID-19 Antigen Test.

| no | substances | Concentration | |
|---|---|---|---|
| 1 | Zanamivir | 5 | mg/ml |
| 2 | lumefantrine | 50 | UM |
| 3 | Doxycycline hyclate | 70 | uM |
| 4 | Quinine | 150 | uM |
| 5 | Lamivudine | 1 | mg/ml |
| 6 | Ribavirin | 1 | mg/ml |
| 7 | Acetaminophen | 200 | UM |
| 8 | Acetylsalicylic acid | 3.7 | mM |
| 9 | Ibuprofen | 2.5 | mM |
| 10 | Mupirocin | 10 | mg/ml |
| 11 | Tobramycin | 5 | ug/ml |
| 12 | Erythromycin | 81.6 | uM |
| 13 | Ciprofloxacin | 31 | uM |
| 14 | Synephrin | 10% (v/v) | |
| 15 | Budesonide | 10% (v/V) | |
| 16 | Sodium Cromoglycate | 20 | mg/ml |
| 17 | Olopatadine Hydrochloride | 10 | mg/ml |
| 18 | Benzocaine | 5% (v/v) | |
| 19 | flurbiprofen | 5% (w/v) | |
| 20 | Mucin: bovine submaxillary | 100 | ug/ml |
| | gland, type I-S | | |
| 21 | Biotin | 100 | ug/ml |

### Example 5-Clinical Performance Evaluation of the Good Plus SARS-COV-2 Antigen Test Utilizing Latex Beads and Biotin-Polystreptavidin Versus KFDA Approved RT-PCR Test

### (South Korea)

[Example 5-1] Sixty remnant specimens (40 positive, 20 negative as diagnosed by GG SARS-COV-2 Real-Time RT PCR that got emergency approval from the Korea FDA) that were nasopharyngeal samples carried in viral transport media from patients suspected of Coronavirus Disease 2019 (SARS-COV-2) were used in the evaluation. The evaluation fulfilled the following specifications: know date of symptom, sample collection date, confirmed date of diagnosis and were preserved at 80° C. for less than a year after quick-freeze at 65° C. in 90 μL of buffer for more than 30 minutes were used to get diagnostic sensitivity and specificity of the systems and methods herein.

Either fresh samples of 100 ul or samples were preserved in −80° C. after deactivation at 65° C. for more than 30 minutes. Then, 10 ul of sample was mixed into 90 ul buffer sample buffer then applied to 100 ul extraction buffer was used for the reaction and visual inspection was carried out.

The results, as presented in Table 3 Table 3 and Figured 8, 9, 10, were directed to the comparison study of the performance of current LFA immunochromatography method using Carboxyl Latex Beads and Biotin-Polystreptavidin compared to Korean FDA approved RT-PCR; where 19 of the 20 of the nasopharyngeal samples the previously tested positive for Korean FDA approved RT-PCR tested positive for the current LFA immunochromatography method using Carboxyl Latex Beads and Biotin-Polystreptavidin by visual inspection using a scoring card. This showed a high sensitivity of 95% and specificity of 100% (Table 3.). More specifically, Table 3 is the Clinical performance evaluation of the Good Ag COVID-19 test utilizing Latex Beads and Biotin-Polystreptavidin in comparison with the GG SARS-COV-2 QPlex Real-Time RT PCR.

TABLE 3
LFA Results in 30 clinical samples (Nasopharyngeal swabs)

| | | | | | | Latex | |
|---|---|---|---|---|---|---|---|
| | | RT-PCR | | | | microsphere | |
| No | sample ID | RdRP | N | ORF1ab | B-actin | Score | Result |
| 1 | HMN461714 | 12.56 | 11.45 | 13.59 | 14.53 | 7 | Positive |
| 2 | HMN461715 | 11.38 | 10.83 | 12.48 | 19.06 | 10 | Positive |
| 3 | HMN461726 | 20.28 | 20.52 | 21.86 | 19.24 | 9 | Positive |
| 4 | HMN462396 | 19.01 | 18.86 | 20.72 | 17.60 | 2 | Positive |
| 5 | HMN462404 | 17.56 | 15.12 | 18.83 | 16.52 | 4 | Positive |
| 6 | HMN467816 | 20.52 | 26.26 | 22.87 | 20.93 | 4 | Positive |
| 7 | HMN467879 | 24.83 | 25.91 | 26.74 | 20.43 | 5 | Positive |
| 8 | HMN467893 | 14.00 | 14.64 | 15.38 | 36.89 | 10 | Positive |
| 9 | HMN467899 | 12.10 | 11.50 | 12.87 | 16.45 | 10 | Positive |
| 10 | HMN467901 | 18.36 | 15.87 | 19.94 | 17.95 | 5 | Positive |
| 11 | HMN467907 | 14.75 | 14.35 | 16.37 | 19.60 | 8 | Positive |
| 12 | HMN467910 | 21.74 | 21.69 | 23.08 | 20.95 | 3 | Positive |
| 13 | HMN467916 | 16.42 | 17.70 | 18.10 | 38.47 | 10 | Positive |
| 14 | HMN467917 | 15.94 | 23.11 | 17.89 | 19.67 | 10 | Positive |
| 15 | HMN467928 | 16.35 | 17.93 | 17.97 | 22.60 | 8 | Positive |
| 16 | HMN467989 | 22.25 | 23.33 | 23.65 | 21.18 | 9 | Positive |
| 17 | HMN467995 | 23.75 | 26.50 | 25.52 | 25.78 | 5 | Positive |
| 18 | HMN468002 | 18.74 | 21.73 | 20.63 | 24.03 | 10 | Positive |
| 19 | HMN468003 | 25.56 | 27.23 | 27.23 | 25.45 | 6 | Positive |
| 20 | HMN467895 | 32.31 | 32.67 | 33.15 | 37.61 | 0 | Negative |

No of Positive Samples 19 19
* scored by eye exam in comparison with provided score card
** N/A: Not Applicable
Total no# of samples: 20

TABLE 4
The diagnostic sensitivity and specificity of Example 1 using
CL-B/PS LFA assay

| | | Standard test (PCR) | | |
|---|---|---|---|---|
| | | Positive | Negative | Total |
| Carboxyl Latex Bead | Positive | 19 | 0 | 19 |
| immunochromatography | | | | |
| (CL-B/PS LFA) | Negative | 1 | 40 | 41 |
| Total | | 20 | 40 | 60 |

| | |
|---|---|
| diagnostic sensitivity | 95% (19/20) |
| | (95% confidence |
| diagnostic specificity | 100.0% (40/40) |
| | (95% confidence |

### Example 5-2—Clinical Performance Evaluation of the Good Plus SARS-COV-2 Antigen Test Utilizing Latex Beads and Biotin-Polystreptavidin as a Self-Administered Rapid Test Versus FDA Approved RT-PCR Test (USA)

Clinical test sites set up in New York, NY was targeted to process test capabilities on nasal swab specimens. Clinical labs set up in Kenilworth, NJ and Seoul, South Korea will be targeted to process test capabilities on nasal swab and nasopharyngeal swab specimens. Standard of care testing was obtained for the reference method. The enrolled population for this clinical investigation included symptomatic and asymptomatic patients that are being tested for COVID-19. Patients provided a signed informed consent form and provide 2 self-performed NS specimens. In the case of children, specimen collection was performed by a parent. The nasal specimen was tested on the Good Ag Rapid COVID-19 Antigen test. The NS specimen is part of clinical care and the results for the testing will be pulled from the lab report performed with Euroimmune at RDX biosciences as well as have Ct values obtained from staff.

The results, as presented in Table 5 and 6 were directed to the comparison study of the performance of current LFA immunochromatography method using Carboxyl Latex Beads and Biotin-Polystreptavidin compared to FDA approved RT-PCR

TABLE 5
Good Ag Antigen Test Kit vs. EUA PCR Test Comparator

| | | EUA PCR Test Kit² | | |
|---|---|---|---|---|
| | | Positive | Negative | Total |
| Good Ag Antigen | Positive | 27 | 0 | 27 |
| Test Kit¹ | Negative | 4 | 123 | 127 |
| Total | | 31 | 123 | 154 |

¹Good Ag COVID-19 Antigen Test: Predicted Condition
²EURO RealTime SARS-CoV-2 test Kit: True Condition

PPA and NPA was calculated using the definitions in the equations below, which can be found in CLSI EP12: User Protocol for Evaluation of Qualitative Test Performance: protocol describes the terms positive percent agreement (PPA) and negative percent agreement (NPA):

$$Sensitivity = a / ( a + b ) \times 100$$

$$Specificity = d / ( b + d ) \times 100$$

$$P P A = a / ( a + c ) \times 100$$

$$N P A = d / ( c + d ) \times 100$$

Accuracy=Sensitivity×Prevalence+Specificity×(1−Prevalence)

The point estimates and exact 95% confidence intervals were reported for PPA and NPA using the exact binomial method.

### Raw Data:

| | | | Good Ag |
|---|---|---|---|
| Sample | PCR results | | Covid-19 |
| Accession | IC | N | Results |
| 2203100727 | 26.968 | Undetermined | − |
| 2203100728 | 31.551 | Undetermined | − |
| 2203101019 | 30.885 | Undetermined | − |
| 2203101021 | 27.033 | 15.051 | + |
| 2203100785 | 30.896 | Undetermined | − |
| 2203100788 | 31.443 | Undetermined | − |
| 2203100883 | 29.778 | Undetermined | − |
| 2203100886 | 31.442 | Undetermined | − |
| 2203100730 | 28.778 | Undetermined | − |
| 2203100889 | 32.448 | Undetermined | − |
| 2203100791 | 30.352 | Undetermined | − |
| 2204060786 | 28.366 | 39.272 | + |
| 2203100793 | 29.317 | Undetermined | − |
| 2203100794 | 30.988 | Undetermined | − |
| 2203100891 | 32.857 | Undetermined | − |
| 2203100796 | 30.402 | Undetermined | − |
| 2203100798 | 32.277 | Undetermined | − |
| 2203100799 | 30.64 | Undetermined | − |
| 2203100892 | 31.166 | Undetermined | − |
| 2203100895 | 29.379 | Undetermined | − |
| 2203100732 | 31.909 | Undetermined | − |
| 2203100801 | 30.799 | Undetermined | − |
| 2204060810 | 25.837 | Undetermined | − |
| 2203100806 | 27.338 | Undetermined | − |
| 2203100809 | 30.312 | Undetermined | − |
| 2203100734 | 27.236 | 26.854 | + |
| 2204060709 | 28.74763298 | 37.60017776 | + |
| 2204060713 | 26.96655083 | 28.00911522 | + |
| 2204060764 | 28.48101616 | 17.09230995 | + |
| 2203100738 | 29.979 | Undetermined | − |
| 2203100739 | 32.134 | Undetermined | − |
| 2203100741 | 30.977 | Undetermined | − |
| 2204060733 | 28.40563393 | Undetermined | − |
| 2204060819 | 34.61365891 | Undetermined | − |
| 2203100747 | 30.119 | Undetermined | − |
| 2204060720 | 28.44776344 | 14.48861313 | + |
| 2204060802 | 28.69613838 | Undetermined | − |
| 2203100817 | 30.644 | Undetermined | − |
| 2203100818 | 30.567 | Undetermined | − |
| 2203100821 | 29.45 | Undetermined | − |
| 2204060715 | 32.93 | 28.473 | + |
| 2203100748 | 29.666 | Undetermined | − |
| 2203100749 | 31.417 | Undetermined | − |
| 2203100756 | 32.71 | Undetermined | − |
| 2203100758 | 31.243 | Undetermined | − |
| 2203100759 | 30.043 | Undetermined | − |
| 2203100822 | 30.81 | Undetermined | − |
| 2203100971 | 31.827 | Undetermined | − |
| 2204060757 | 28.62850761 | Undetermined | − |
| 2204060718 | 28.54040337 | Undetermined | − |
| 2204060833 | 28.71381378 | 22.03205681 | + |
| 2204060785 | 34.023 | 15.858 | − |
| 2204060731 | 27.8798542 | 38.65799713 | + |
| 2203100974 | 32.166 | Undetermined | − |
| 2204060823 | 28.20266151 | 41.63447189 | − |
| 2204060724 | 28.79990387 | 14.83823204 | + |
| 2204060735 | 31.59833908 | Undetermined | − |
| 2204060753 | 29.45583344 | Undetermined | − |
| 2204060745 | 28.01458931 | Undetermined | − |
| 2204060743 | 28.19228363 | Undetermined | − |
| 2204060777 | 22.18107986 | 13.57145882 | + |
| 2204060752 | 28.38672256 | Undetermined | − |
| 2204060820 | 26.88361359 | 11.12584686 | + |
| 2203100975 | 31.14 | Undetermined | − |
| 2204060783 | 29.86561966 | 41.60478973 | − |
| 2204060830 | 28.42406845 | Undetermined | − |
| 2204060805 | 29.93679047 | 33.9719162 | + |
| 2204060747 | 27.8137989 | 20.82567978 | + |
| 2204060768 | 28.58494759 | Undetermined | − |
| 2204060716 | 28.30427742 | Undetermined | − |
| 2204060762 | 28.21657753 | Undetermined | − |
| 2204060799 | 27.67358589 | 21.64191246 | + |
| 2204060727 | 34.65416336 | Undetermined | − |
| 2203100977 | 32.134 | Undetermined | − |
| 2203100978 | 31.65 | 38.618 | + |
| 2203100979 | 30.764 | Undetermined | − |
| 2204060813 | 25.837 | 33.129 | + |
| 2204060736 | 29.05265045 | 36.26053619 | + |
| 2204060722 | 28.58236504 | 40.01940536 | + |
| 2204060846 | 28.61204147 | Undetermined | − |
| 2204060750 | 28.42567825 | Undetermined | − |
| 2204060741 | 28.27330399 | Undetermined | − |
| 2204060842 | 30.2254982 | 21.64840508 | + |
| 2204060774 | 30.539 | 25.148 | − |
| 2203100980 | 30.791 | Undetermined | − |
| 2203100896 | 30.287 | Undetermined | − |
| 2203100824 | 30.99 | Undetermined | − |
| 2203100984 | 30.877 | 15.354 | + |
| 2203100986 | 31.479 | Undetermined | − |
| 2203100897 | 30.46 | Undetermined | − |
| 2203100987 | 29.602 | Undetermined | − |
| 2203100990 | 31.724 | Undetermined | − |
| 2203100830 | 30.292 | Undetermined | − |
| 2203100833 | 31.268 | Undetermined | − |
| 2203100835 | 32.152 | Undetermined | − |
| 2204060836 | 27.43927002 | 21.15965271 | + |
| 2203100899 | 30.629 | Undetermined | − |
| 2203101023 | 30.7 | Undetermined | − |
| 2203100838 | 31.33 | Undetermined | − |
| 2203100991 | 32.527 | Undetermined | − |
| 2203100993 | 32.989 | Undetermined | − |
| 2203101025 | 29.637 | Undetermined | − |
| 2203100995 | 32.491 | Undetermined | − |
| 2203100996 | 31.811 | Undetermined | − |
| 2203100998 | 30.166 | Undetermined | − |
| 2203100839 | 33.419 | Undetermined | − |
| 2203101026 | 27.941 | Undetermined | − |
| 2203100999 | 30.721 | Undetermined | − |
| 2203101001 | 30.461 | Undetermined | − |
| 2203100845 | 32.164 | Undetermined | − |
| 2203101027 | 30.615 | Undetermined | − |
| 2203101031 | 30.315 | Undetermined | − |
| 2203101033 | 29.624 | Undetermined | − |
| 2203101036 | 30.275 | Undetermined | − |
| 2203101039 | 30.946 | Undetermined | − |
| 2203100846 | 30.507 | Undetermined | − |
| 2203101004 | 30.98816681 | Undetermined | − |
| 2203101005 | 30.205 | Undetermined | − |
| 2203101040 | 29.73 | Undetermined | − |
| 2203101006 | 28.248 | Undetermined | − |
| 2203100902 | 29.415 | Undetermined | − |
| 2203100849 | 29.254 | Undetermined | − |
| 2203101042 | 29.902 | Undetermined | − |
| 2203100852 | 24.274 | Undetermined | − |
| 2203101043 | 32.449 | 17.632 | + |
| 2204060824 | 40.32343674 | 28.86277199 | + |
| 2203101049 | 26.991 | Undetermined | − |
| 2203100853 | 29.913 | Undetermined | − |
| 2203101008 | 31.212 | Undetermined | − |
| 2203101050 | 29.913 | Undetermined | − |
| 2203100783 | 30.833 | Undetermined | − |
| 2203101010 | 31.238 | Undetermined | − |
| 2203100858 | 29.607 | Undetermined | − |
| 2203100860 | 32.602 | Undetermined | − |
| 2203100863 | 30.837 | Undetermined | − |
| 2203101011 | 30.75 | Undetermined | − |
| 2203101052 | 31.995 | Undetermined | − |
| 2203101013 | 31.499 | Undetermined | − |
| 2203101053 | 29.983 | Undetermined | − |
| 2203100866 | 26.193 | 24.655 | + |
| 2203100906 | 30.739 | Undetermined | − |
| 2203101056 | 30.151 | Undetermined | − |
| 2203100868 | 31.073 | Undetermined | − |
| 2203100870 | 30.526 | Undetermined | − |
| 2203101059 | 30.635 | Undetermined | − |
| 2203100872 | 29.752 | Undetermined | − |
| 2203101016 | 30.741 | Undetermined | − |
| 2203100874 | 30.432 | Undetermined | − |
| 2203101062 | 29.975 | Undetermined | − |
| 2203100878 | 28.919 | Undetermined | − |
| 2203101017 | 30.707 | Undetermined | − |
| 2203101064 | 30.151 | Undetermined | − |
| 2204060808 | 32.20455551 | 36.81802368 | + |
| 2203100882 | 29.693 | Undetermined | − |

| Product group | In vitro diagnostic medical device | | |
|---|---|---|---|
| Product name | High-risk infectious agent | Item | K05030.01 |
| | test reagent immunological | classification | |
| Model name | Good Ag COVID-19 | Class | Class 3 |
| | antigen test | | |
| Manufacturer | PRECISION BIO Co., | | |
| | Ltd./Cellgenemedix Corp | | |
| Country | Republic of Korea | | |

### Result:

| Statistic | Value | 95% CI |
|---|---|---|
| Sensitivity | 87.10% | 70.17% to 96.37% |
| Specificity | 100.00% | 97.05% to 100.00% |
| Disease prevalence (*) | 20.13% | 14.11% to 27.34% |
| Positive Predictive Value (*) | 100.00% | 87.23% to 100.00% |
| Negative Predictive Value (*) | 96.85% | 92.49% to 98.71% |
| Accuracy (*) | 97.40% | 93.48% to 99.29% |

### Example 6—Production Manual of the Good Plus SARS-COV-2 Antigen Test Utilizing Latex Beads and Biotin-Polystreptavidin

### 1. Purpose

The purpose of this Product Standard is to prescribe overall details including product specifications, characteristics, processes, tests and inspections, and labeling in order to satisfy the requirements for the Good Ag COVID-19 antigen test kit manufactured by our company.

### 2. Scope of Application

### 3. Shape and Structure

### 1) Background

There are four genera (alpha, beta, gamma, delta) in the coronaviridae, and alpha and beta are known to infect humans and animals. As the name of the virus, the virus has a crown-shaped spherical shape characterized by an external spike protein. Before this novel virus was known, there were six known coronaviruses that infect humans. There were four types causing the common cold (229E, C43, NL63, HKU1) and two types causing severe pneumonia (SARS-COV, MERS-COV). SARS-COV-2 identified this time is the third type that causes severe pneumonia.

This product is for checking the presence or absence of coronavirus antigen in nasal, oropharyngeal, or nasopharyngeal swabs collected from a person with symptoms of recent onset (less than 6 days) of coronavirus infection symptoms through immunochromatography.

### 2) Appearance

### 3) Components and Characteristics of the Product

### (1) Components of the Product

| Components | Appearance Features |
|---|---|
| 1. Device | It is a white rectangular plastic cassette. |
| | There is a circular sample drop site att |
| | he bottom of both sides, and the positions |
| | of the control line (c) and the test line (T) |
| | are marked on the display window. |
| 2. Sample | It is a colorless and transparent liquid |
| extract | contained in a colorless transparent tube. |
| 3. User Manual | Paper print |
| 4. Swap | A sponge is attached to the end of the |
| | plastic material. |
| 5. Nozzle cap | It is a colorless and transparent plastic |
| | material. |

### (2) Characteristics

### Principle

Good Ag COVID-19 Test has a control line and a test line on the membrane. The control line is coated with anti-chicken IgY antibody, and the test line is coated with streptavidin. No line appears on the result window before sample input. The high surfactant in the swab extraction solution elutes the antigenic proteins in the virus in the extraction solution. When the extracted sample is instilled, if SARS-CoV-2 antigen is present in the sample, it binds to the anti-COVID-19 mAb bound to the biotin pad to form an antigen-antibody complex. This complex moves along the membrane to the latex bonding pad through capillary phenomenon to form the antibody biotin-antigen-antibody latex complex. The formed complex moves along the membrane to the test line, binds to the streptavidin fixed to the test line, and displays a red line in the result confirmation window. If the SARS-COV-2 antigen is not present in the sample, no red line is displayed on the test line, and the control line is displayed in red if the test procedure is performed correctly

### 4. Raw Materials

1) Quantity and specifications of raw materials

| | | Purpose of | raw material or | Amount | | |
|---|---|---|---|---|---|---|
| SN | Name | mixing | ingredient | (for 1 test) | Standard | Note |

| | | | | | | | |
|---|---|---|---|---|---|---|---|
| 1 | Device | Sample pad | Support | Cotton Fiber | 4.0 × 13 × 01. | mm | Own |
| | | | | | | | Standard |
| | | Bonding pad | Main | carboxylic latex beads | 0.5 ± 0.05 | μg | Own |
| | | | ingredient | conjugated mouse | | | Standard |
| | | | Main | carboxylic latex beads | 0.5 ± 0.05 | μg | Own |
| | | | ingredient | conjugated chicken | | | Standard |
| | | | Main | Biotin Conjugated | 56 ± 5 | ng | Own |
| | | | ingredient | mouse anti-COVID-19 | | | Standard |
| | | | Support | Glass fiber | 4.0 × 7.0 × 0.1 | mm | Own |
| | | | | | | | Standard |
| | | Control line | Main | mouse anti-Chicken | 0.5 ± 0.05 | μg | Own |
| | | | ingredient | IgY mAb | | | Standard |
| | | Test line | Main | Polystreptavidin R | 0.16 ± 0.16 | μg | Own |
| | | | ingredient | | | | Standard |
| | | Membrane | Support | Nitrocellulose | 4.0 × 25.0 × 0.1 | mm | Own |
| | | | | membrane | | | Standard |
| | | Absorbent pad | Support | Cotton Fiber | 4.0 × 18.0 × 0.1 | mm | Own |
| | | | | | | | Standard |
| | | Exterior plastic | Support | PVC | 4.0 × 60.0 × 0.2 | mm | Own |
| | | | | | | | Standard |

| | | | | | | |
|---|---|---|---|---|---|---|
| 2 | Sample extract | Surfactant | IGEPAL CA-630 | 1% | Own | 400 ul |
| | | | | | Standard | |

| | | | |
|---|---|---|---|
| preservative | Sodium azid | 0.1% | Own |
| | | | Standard |
| Buffer | 1M Tris-Hcl | 10% | Own |
| | | | standard |

### 5. Effect

This is an in vitro diagnostic medical device for diagnosing coronavirus disease of 2019 (COVID-19) by detecting the presence of nucleic acid (RNA) of novel coronavirus (SARS-Cov-2) in human upper respiratory (H|pharynx or oropharyngeal swab or aspiration or lavage) or lower respiratory samples (sputum, bronchoalveolar lavage, tracheal aspirate).

### Manufacturing Method

### 1) Manufacturing Process

Membrane & pad manufacturing table

| Membrane | Pad | | | Sample | Absorbent |
|---|---|---|---|---|---|
| Coating | Pretreatment | Latex pad | Biotin pad | pad | Pad |
| Raw | Raw | Raw | Raw | Raw | Raw |
| material | material | material | material | material | material |
| preparation: | preparation: | preparation: | preparation: | preparation: | preparation: |
| 1) Plastic | 1) Glass | 1) Latex | 1) | Cellulose | Cellulose |
| card | fiber (Grade | target | Biotinylated | Paper | Paper |
| (60 mm × 300 | 8964) pad | antibody | antibody | Cut to | Cut to |
| mm, A: 0- | 200 mm × 30 | conjugated | solution | 300mm X | 300mm X |
| 14 mm, | 0 mm size | solution | 2) 2X CBD | 13mm. | 13mm. |
| B: 14-39.5 | 2) 30 ml pad | (covid-19) | (5% casein | | |
| mm, | pretreatment | 2) Latex- | amino acid, | | |
| C39.5- | solution | Chicken IgY | 20 mM | | |
| 45 mm, | Apply 30 ml | Conjugated | EDTA, 1% | | |
| D: 45-60 mm | of pad | Solution | trehalose, | | |
| from top) | pretreatment | 3) 2X CDB, | 0.1% | | |
| 2) | solution | 5% Casein | sodium azide | | |
| 0.5 mg/ml | evenly to | from bovine | in 20 mM | | |
| Poly- | the pad | milk, 10% | boric acid | | |
| streptavidin | Dry for 4 | Tween-20 | pH 8.5), 5% | | |
| (test line) | hours at | 4) | Casein from | | |
| and | 40° C. in a | Pretreatment pad | bovine milk | | |
| 1.5 mg/ml | dry oven | 7 mm × 300 mm | 10% | | |
| Anti- | | Latex | Tween-20 | | |
| Chicken | | dispensing | Grade 8964 | | |
| IgY using | | solution: | pad | | |
| 50 mM | | Dilute the | 7 mm × 300 mm | | |
| sodium | | latex | Biotin | | |
| phosphate | | conjugated | dispensing | | |
| buffer | | solution | solution: | | |
| (Ph7.5) | | (Chicken | Dilute | | |
| Remove the | | IgY and | biotinylated | | |
| cover tape | | covid-19) to | antibody to | | |
| from the | | the target | target | | |
| plastic card | | concentration. | concentration | | |
| part B, and | | Apply 980 ul | Apply 1.12 ml | | |
| then attach | | of latex | of biotin | | |
| the membrane. | | dispensing | dispensing | | |
| Check the | | solution | solution | | |
| position of | | evenly on the | evenly on | | |
| the | | pretreatment | 8994 pad | | |
| dispensing | | pad | 5 m Torr, | | |
| nozzle on | | 5 m Torr, | Maintain at | | |
| the control | | Maintain at | 30° C. (Tray | | |
| line and | | 30° C. (Tray | temperature) | | |
| inspection | | temperature) | and dry for | | |
| line | | and dry for | 3 hours | | |
| Prepare 0.5 | | 3 hours | | | |
| mg/ml poly- | | | | | |
| streptavidin | | | | | |
| (test line) | | | | | |
| and 1.5 | | | | | |
| mg/ml Anti- | | | | | |
| Chicken IgY | | | | | |
| (control line) | | | | | |
| Apply | | | | | |
| 0.8 ul/cm | | | | | |
| test line on | | | | | |
| the top | | | | | |
| 30 mm of | | | | | |
| the membrane | | | | | |
| and 0.8 ul/cm | | | | | |
| control line | | | | | |
| on 25 mm | | | | | |
| Dry at 37° C. | | | | | |
| for 3 hours. | | | | | |

Lamination Table

| | | | | | | |
|---|---|---|---|---|---|---|
| Raw material | Remove the | Remove the | Remove the | Attach the | Visual | Attach the |
| preparation: | cover tape from | cover tape from | cover tape from | plastic pad | inspection* | label to the |
| 1) Membrane pad: | the plastic pad | the plastic pad | the plastic pad | to the | | back of the |
| Attached to | part A and | part C and | part D and | biotin pad by | | plastic pad. |
| 14.5~39.5 mm | attach it to | attach it to | attach it to | overlapping | | |
| (25 mm) from | the membrane | the membrane | the membrane | it by 3 mm | | |
| the top of the | by overlapping | by overlapping | by overlapping | or to the | | |
| plastic card, | it by 3.5 mm. | it by 1.5 mm. | it by 3 mm. | bottom of the | | |
| and arrange | | | | plastic card. | | |
| regularly at | | | | | | |
| 25 mm from the | | | | | | |
| top of the | | | | | | |
| control line | | | | | | |
| and 30 mm from | | | | | | |
| the bottom of | | | | | | |
| the test line | | | | | | |
| 2) Absorbent pad: | | | | | | |
| 0~14 mm (18 mm) | | | | | | |
| from the top of | | | | | | |
| the plastic card | | | | | | |
| 3) Latex pad: | | | | | | |
| 39.5~45 mm(7 mm) | | | | | | |
| from the top of | | | | | | |
| the plastic card | | | | | | |
| 4) Biotin pad: | | | | | | |
| 45~60 mm(7 mm) | | | | | | |
| from the top of | | | | | | |
| the plastic card | | | | | | |
| 5) Sample pad: | | | | | | |
| 47~60 mm(13 mm) | | | | | | |
| from the top of | | | | | | |
| the plastic card | | | | | | |
| Plastic pad | | | | | | |

A: Absorbent pad
B: Membrane pad −> Made in the membrane manufacturing process
C: Latex pad
D: Biotin pad + sample pad

Strip & Pouch Packaging

| | | | | |
|---|---|---|---|---|
| Strip | Plastic | Pouch | Packaging | Finished |
| cutting | housing | assembly | | product |
| | assembly | | | inspection* |

| | | |
|---|---|---|
| Cut the dressing | Semi-finished product | Check the condition |
| plate at 4 mm | inspection* | of the upper and |
| intervals with a strip | Securely fix the upper | lower plates of the |
| cutter. | and lower plates using | plastic housing. |
| Insert it correctly into | automated equipment. | Put the device and |
| the strip attachment | Raw material | moisture absorbent |
| structure so that | packaging | in the pouch. |
| there are no gaps. | | Finished product |
| After adjusting the | | inspection* |
| temperature and | | |
| speed of the | | |
| packaging machine, | | |
| proceed with sealing | | |
| and attach the label. | | |

| | |
|---|---|
| 3) | Finished product packaging |
| | After putting 25 sample extracts into the pouch, seal it and |
| | attach the label. |
| | Put 25 cotton swabs in the pouch and attach the label. |
| | Put 25 nozzle caps into the pouch. |

How to use

| 1) | Specimen preparation and storage method Nasopharyngeal | |
|---|---|---|
| | swab | |
| | a. | Gently push the sterile swab along the nasal septum to |
| | | move it to the back of the nasopharynx, and then rotate |
| | | it 3-4 times on the surface near the back of the |
| | | nasopharynx. |
| | b. | Carefully withdraw the sterile swab out of the nostril. |
| | c. | After the nasopharyngeal swab is smeared, put the swab |
| | | in the extraction solution and dilute it at least 5 times. |
| | d. | Slowly withdraw the swab from the extraction solution |
| | | tube and press both sides to drain the sample mixture |
| | | absorbed in the swab sufficiently. |
| | e. | Close the extraction solution tube with the nozzle cap. |
| | f. | Collected samples should be tested with 72 hours under |
| | | refrigerated storage conditions. If it takes longer than |
| | | that, keep it frozen at −20° C. or lower. |
| 2) | Preparations before inspection | |
| | a. | Read the instuctions and procedures in this user manual |
| | | carefully. |
| | b. | Check the expiry date printed on the back of the pouch. |
| | c. | Use only products that have not passed their expiry date. |
| | d. | Check the color label of the device for inspection and |
| | | desiccant in the pouch. |
| 3) | Inspection method | |
| | a. | Before testing, take out the packaged testing device and |
| | | the diluted sample to room temperature. |
| | b. | Just before the test, open the pouch, take out the test |
| | | device, and place it on a flat surface. |
| | c. | Hold the extraction solution buffer tube upside down |
| | | and put 3 drops of the sample mixture onto the sample |
| | | drop site of the test device. |
| | d. | Check the test results up to 20 minutes. Results after |
| | | 20 mintues are unreliable. |
| | e. | Avoid repeated freezing/thawing of samples. |
| 4) | Result judgment | |
| | a. | Negative: The band appears only at the control line (C) |
| | | position |
| | b. | Positive: The band appears at both the control line (C) |
| | | and the test line (T) |
| | c. | Retest: No bands or bands only on the test line. |
| | 8. | Test standards |
| | 8.1 | Recipt Inspection |
| | 8.1.1 | Chicken IgY, Anti-corona NP |
| 1) | Visual inspection | |
| | \textcircled{1} | Replace with external report. |
| 8.1.2. | Sample extract | |
| | \textcircled{1} | Check for foreign substances, quantity, and capacity |
| | | Check the label attached to the compenent and indications. |
| 2) | Performance inspection | |
| | \textcircled{1} | Dilute the positive standard substance diluted to a |
| | | concentration of 10 ug/ml with 270 ul of sample |
| | | diluted buffer. |
| | | Mix 30 ul of the standard substance diluted to a |
| | | concentration of 10 ug/ml with 270 ul of sample |
| | | diluted buffer. |
| | \textcircled{3} | Prepare 300 ul of the sample extract and drop 100 |
| | | ul 3 times on the device. (negative confirmation) |
| | \textcircled{4} | Drop 100 μl of the sample 3 times on the drop site |
| | | at the bottom of the test device. (positive confirmation) |
| | \textcircled{5} | Deciphering time is 20 minutes, and deciphering is |
| | | not performed after 30 minutes. |
| 8.2 | Semi-finished product inspection | |
| 1) | Visual inspection | |
| | \textcircled{1} | Check for foreign substances |
| | \textcircled{2} | Check the strip and device mark. |
| | \textcircled{3} | Check for scratches and damage. |
| | \textcircled{4} | Check the quantity. |
| 2) | Performance inspection | |
| | \textcircled{1} | Prepare a positive standard substance. |
| | \textcircled{2} | Dilute the positive standard substance using a |
| | | diluent solution. (2 ug/ml, 1 ug/ml, 0.2 ug/ml, |
| | | 0.1 ug/ml) |
| | \textcircled{3} | Mix 30 ul of standard substances diluted to its |
| | | respective concentration into 270 ul of sample |
| | | diluted buffer. |
| | \textcircled{4} | Prepare 300 ul of the sample extract and drop |
| | | 100 ul 3 times on the device. (negative confirmation) |
| | \textcircled{5} | Drop 100 μl of the sample 3 times on the drop site at |
| | | the bottom of the test device. (positive confirmation) |
| | \textcircled{6} | Deciphering time is 20 minutes, and deciphering is |
| | | not performed after 30 minutes. |
| 8.3 | Finished product inspection | |
| 1) | Visual inspection | |
| | \textcircled{1} | Check if the packaging material is damaged. |
| | \textcircled{2} | Check whether the packaging material is deformed. |
| | \textcircled{3} | Check the label attached to the component and |
| | | indications. |
| | \textcircled{4} | Check the kit components in the packaging. |
| 2) | Performance inspection | |
| | \textcircled{1} | Prepare a positive standard substance. |
| | \textcircled{2} | Dilute the positive standard substance using a |
| | | diluent solution. (10 ug/ml, 1 ug/ml, 0.1 ug/ml) |
| | \textcircled{3} | Mix 30 ul of standard substances diluted to its respective |
| | | concentration into 270 ul of sample diluted buffer. |
| | \textcircled{4} | Prepare 300 ul of the sample extract and drop 100 ul |
| | | 3 times on the device. (negative confirmation) |
| | \textcircled{5} | Drop 100 μl of the sample 3 times on the drop site at |
| | | the bottom of the test device. (positive confirmation) |
| | \textcircled{6} | Deciphering time is 20 minutes, and deciphering is |
| | | not performed after 30 minutes. |

Put \textcircled{2} \textcircled{3} and 25 individually wrapped devices in the box, and put the
instuction manual.
No. Inspection Inspection method Inspection standards Remarks

| | |
|---|---|
| 1 | Incoming goods inspection Chicken IgY Visual Check the presence |
| | of foreign substances, capacity and quantity, labels and indications |
| | Refer to Import Inspection Standard Report (F805-01) |
| | Performance Replace with external report |
| 2 | Anti-cornona NP Visual Check the presence of foreign |
| | substances, capacity and quantity, labels and indications Refer to Import |
| | Inspection Standard Report (F805-01) |
| 3 | Sample diluted buffer Visual Check for foreign substances and |
| | capacity, Colorless Refer to Import Inspection Standard Report (F805-01) |
| | Performance Positive in high concentration and negative in sample |
| | extract |
| 6 | Semi-finished product inspection Antigen membran & latex |
| | conjugated pad Visual Check for foreign substances, scratches and |
| | damage, quantity, strips and device marks Refer to Semi-finished |
| | Product Inspection Standard Report (F805-03) |
| | Performance Positive at high, medium, and low concentrations, and |
| | gegative in sample extract |
| 7 | Finished product inspection Visual Check the packaging material |
| | for damage, deformation, labeling, and components. Refer to Final |
| | Product Inspection Standard Report (F805-02) |
| | Performance Positive at high, middle, and low concentrations, and |
| | negative in sample extract |
| 9. | Packaging unit and packaging method |
| 9.1 | Packaging method |

SN Name Form Characteristics Remarks

| | |
|---|---|
| 1 | Device Pouch packaging Plastic device in the pouch |
| 2 | Sample extract Pouch packaging Colorless transparent liquid in a |
| | transparent tube in the pouch |
| 3 | Sterile swab Plastic zipper big A sponge is attached to the end of |
| | the plastic material and individually packaged. |
| 4 | Nozzle cap Plastic zipper bag A sponge is attached to a colorless |
| | and transparent plastic material. |
| 5 | User manual Prints Printed instructions for use |
| 9.2 | Packaging unit |
| | GG-CAG-01 GG-CAG-02 GG-CAG-03 |
| | Packaging unit |
| | Raw material 10 Test/kit 25 Test/kit 50 Test/kit |
| | Device 10 25 50 |
| | Sample diluted solution 400 ul × 10 400 ul × 25 400 ul × 50 |
| 10. | Storage method and expiration date |
| | SN Name Status Storage condition Use period (from the date of |
| | production) Remarks |
| 1 | Device Unopened Room temperature 12 months Finished product |
| | (disposable) |
| 2 | Sample extract Unopened Room temperature 12 monts Finished product |
| | Opened 2~30° C. 1 month |
| 3 | Sterile swab Unopened Room temperature 12 months Finished product |
| | (disposable) |
| 12. | Details |
| 1 | Item name/model name High-risk infectious agent immunity test |
| | reagent/ |
| | Good Ag COVID-19 antigen test (GG-CAG-01) |
| 2 | Manufacturing license No. In vitro No. 4810 |
| 4 | Purpose of use An in vitro diagnostic medical device that helps |
| | confirm the presence of SARS-CoV-2 infection by qualitative |
| | immunochromatography of the SARS-CoV-2 antigen (Nucleocapsid |
| | antigen) in the upper and lower respitory tract samples of patients |
| | suspected of having a respiratory infection |
| 5 | Expiration period 12 months from the date of manufacture before |
| | opening, and 1 month from the date of manufacture after opening |
| 6 | Manufacturing number and date of manufacture Indicated at the |
| | time of manufacture (refer to label) |
| 7 | Performance and usage Refer to user manual |
| 8 | Precautions for use Refer to user manual |
| 9 | Storage method Store at room temperature (refer to user manual) |
| 10 | Packaging unit Kit |
| 11 | This product is a disposable in vitro diagnostic medical device. |
| 12 | Manufacturer PRECISION BIO Co., Ltd. |
| | 1111-1112, 28, Digital-ro 30-gil, Guro-gu, Seoul, Republic of Korea |
| | Cellgenemedix LLC |
| | 243 Broadway #9188 SMB# 9564, Newark, New Jersey, 07104 |
| | Cellgenmedix Corp |
| | 1113, 28, Digital-ro 30-gil, Guro-gu, Seoul, Republic of Korea |

*This product cannot differentiate between SARS-CoV-1 and SARS-CoV-2 antigens.

### Example 7—Comparison Test of Diagnostic Kit for SARS-COV-2 Infection Using Latex Beads and Biotin-Polystreptavidin with FDA EUA-Approved Rapid Antigen Test from Competitor Companies Using Gold Nanoparticle

### [Example 7-1] Clinical Sample Real Time RT-PCR and Comparison Result Using 90 Samples

In steps involved in the comparison test of Diagnostic Kit for SARS-COV-2 infection using Latex Beads and Biotin-Polystreptavidin with FDA EUA-approved rapid antigen test from a competitor company using gold nanoparticle are listed below.

There were 90 remnant specimens (30 positive, 60 negative as diagnosed by SARS-COV-2 Real-Time RT PCR that got emergency approval from the Korean FDA) in nasopharyngeal samples carried in viral transport media from patients suspected of Coronavirus Disease 2019 (SARS-COV-2). The following specifications were considered: date of symptom, sample collection date, confirmed date of diagnosis and preserved at −80° C. after inactivation at 65° C. in 90 μL of buffer for more than 30 minutes. The specifications were used to get diagnostic sensitivity and specificity of the systems and methods herein.

The diagnostic Kit for SARS-COV-2 infection using Latex Beads and Biotin-Polystreptavidin (CL-B/PS LFA) and FDA EUA-approved rapid antigen test from a competitor company (BD biosensor, Seoul, South Korea) using gold nanoparticles were carried out in parallel as a comparison test. The comparison test involved: 10 μL of clinical samples that were preserved at −80° C. after inactivation at 65° C. in 90 μL of buffer for more than 30 minutes were mixed with standard extraction buffer, applied to sample well in a total amount of 100 ul, and test results were interpreted by 2 separate experimenter/interpreters according to provided score card. The FDA EUA-approved rapid antigen test from a competitor company using gold nanoparticles used 10 μL of clinical samples mixed with sample buffer 40 ul and 50 ul was applied in the sample well and reacted for 30 minutes. Visual inspection was then carried out by the same 2 separate experimenter/interpreters. These experiments were carried out in a separate lab with no potential conflict of interest.

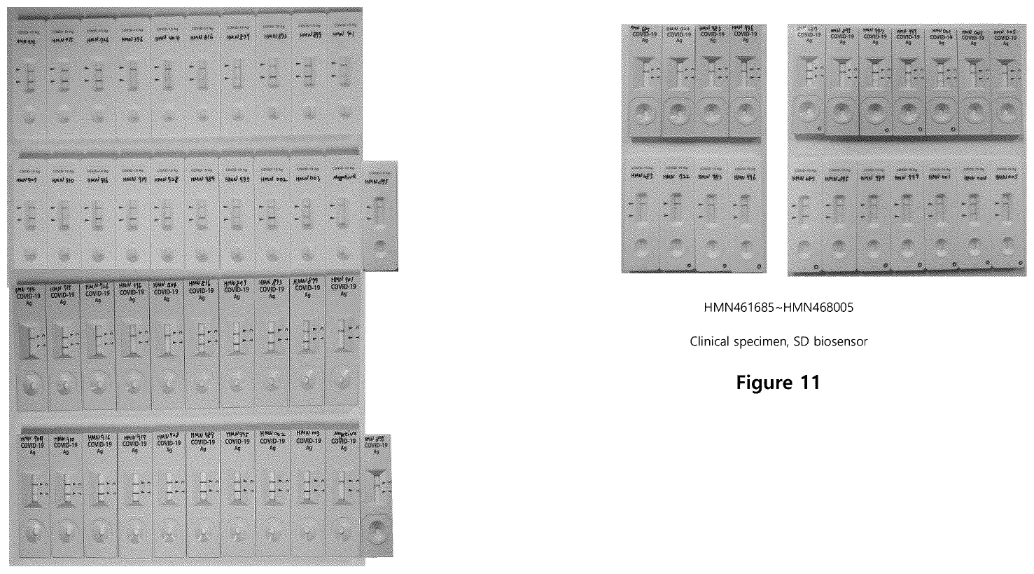

FIG. 11 depicts the results of the comparison test of the diagnostic Kit for SARS-COV-2 infection using Latex Beads and Biotin-Polystreptavidin (CL-B/PS LFA) and FDA EUA-approved rapid antigen test from a competitor company (BD biosensor, Seoul, South Korea) using gold nanoparticles

Of the 30 positive specimens as diagnosed by RT-PCR, 27 were positive by the systems and methods herein (e.g., using Carboxyl Latex Beads Biotin-Polystreptavidin immunochromatography) versus 19 by competitor assay using gold nanoparticles. Of the 60 negative specimens as diagnosed by RT-PCR, 60 were positive by the systems and methods herein (using Carboxyl Latex Beads Biotin-Polystreptavidin immunochromatography) versus 60 by competitor assay using gold nanoparticles (Table 5). Thus, the systems and methods herein (i.e., using Carboxyl Latex Beads Biotin-Polystreptavidin immunochromatography) had a sensitivity of 90% and specificity of 100%, while competitor assay using gold nanoparticles showed sensitivity of 63.3% and a specificity of 100% (Table 6).

TABLE 5
Comparison study between rapid antigen kits using CL-B/PS LFA versus gold
nanoparticles in 30 RT-PCR Positive clinical nasopharyngeal specimens

| | | | | | | latex | | gold | |
|---|---|---|---|---|---|---|---|---|---|
| | | RT-PCR | | | | microsphere | | nanoparticle | |
| No | sample ID | RdRP | N | ORF1ab | B-actin | Score | Result | Score | Result |
| 1 | HMN461714 | 12.56 | 11.45 | 13.59 | 14.53 | 7 | Positive | 0 | Negative |
| 2 | HMN461715 | 11.38 | 10.83 | 12.48 | 19.06 | 10 | Positive | 10 | Positive |
| 3 | HMN461726 | 20.28 | 20.52 | 21.86 | 19.24 | 9 | Positive | 2 | Positive |
| 4 | HMN462396 | 19.01 | 18.86 | 20.72 | 17.60 | 2 | Positive | 0 | Negative |
| 5 | HMN462404 | 17.56 | 15.12 | 18.83 | 16.52 | 4 | Positive | 0 | Negative |
| 6 | HMN467816 | 20.52 | 26.26 | 22.87 | 20.93 | 4 | Positive | 5 | Positive |
| 7 | HMN467879 | 24.83 | 25.91 | 26.74 | 20.43 | 5 | Positive | 9 | Positive |
| 8 | HMN467893 | 14.00 | 14.64 | 15.38 | 36.89 | 10 | Positive | 10 | Positive |
| 9 | HMN467899 | 12.10 | 11.50 | 12.87 | 16.45 | 10 | Positive | 7 | Positive |
| 10 | HMN467901 | 18.36 | 15.87 | 19.94 | 17.95 | 5 | Positive | 0 | Negative |
| 11 | HMN467907 | 14.75 | 14.35 | 16.37 | 19.60 | 8 | Positive | 3 | Positive |
| 12 | HMN467910 | 21.74 | 21.69 | 23.08 | 20.95 | 3 | Positive | 5 | Positive |
| 13 | HMN467916 | 16.42 | 17.70 | 18.10 | 38.47 | 10 | Positive | 9 | Positive |
| 14 | HMN467917 | 15.94 | 23.11 | 17.89 | 19.67 | 10 | Positive | 10 | Positive |
| 15 | HMN467928 | 16.35 | 17.93 | 17.97 | 22.60 | 8 | Positive | 10 | Positive |
| 16 | HMN467989 | 22.25 | 23.33 | 23.65 | 21.18 | 9 | Positive | 10 | Positive |
| 17 | HMN467995 | 23.75 | 26.50 | 25.52 | 25.78 | 5 | Positive | 7 | Positive |
| 18 | HMN468002 | 18.74 | 21.73 | 20.63 | 24.03 | 10 | Positive | 10 | Positive |
| 19 | HMN468003 | 25.56 | 27.23 | 27.23 | 25.45 | 6 | Positive | 9 | Positive |
| 20 | HMN461685 | 37.64 | 34.04 | N/A | 21.70 | 0 | Negative | 0 | Negative |
| 21 | HMN461722 | N/A | 36.21 | N/A | 13.23 | 2 | Positive | 0 | Negative |
| 22 | HMN467983 | 35.76 | N/A | N/A | 22.81 | 2 | Positive | 0 | Negative |
| 23 | HMN467996 | 34.67 | N/A | N/A | 22.01 | 2 | Positive | 0 | Negative |
| 24 | HMN461687 | 31.02 | 30.37 | 33.10 | 18.14 | 9 | Positive | 10 | Positive |
| 25 | HMN467895 | 32.31 | 32.67 | 33.15 | 37.61 | 0 | Negative | 0 | Negative |
| 26 | HMN467997 | 26.48 | 25.71 | 27.43 | 22.91 | 2 | Positive | 2 | Positive |
| 27 | HMN467999 | 27.89 | 28.56 | 28.80 | 21.84 | 2 | Positive | 3 | Positive |
| 28 | HMN468001 | 26.51 | 28.84 | 27.98 | 23.93 | 3 | Positive | 4 | Positive |
| 29 | HMN468004 | 26.77 | 24.61 | 27.37 | 20.49 | 6 | Positive | 0 | Negative |
| 30 | HMN468005 | 33.97 | 33.24 | 39.75 | 21.31 | 0 | Negative | 0 | Negative |

Number of positive samples 27 19
* interpreted by LFA reader scores
** N/A: Not Applicable
Total number of specimens: 30

TABLE 6
The diagnostic sensitivity and specificity in rapid antigen
kits using CL-B/PS LFA in 90 clinical
nasopharyngeal specimens.

| | | Standard test (RT-PCR) | | |
|---|---|---|---|---|
| | | Positive | Negative | Total |
| Carboxyl Latex Bead | Positive | 27 | 0 | 27 |
| Immunochromatography | Negative | 3 | 60 | 63 |
| Total | | 30 | 60 | 90 |

| | |
|---|---|
| diagnostic sensitivity | 90.0% (27/30) (95% confidence |
| | interval: 77.2%~81.7%) |
| diagnostic specificity | 100.0%(60/60) (95% confidence |
| | interval: 99.7%~100%) |

TABLE 7
The diagnostic sensitivity and specificity in control rapid
antigen kits using gold nanoparticles in 90 clinical
nasopharyngeal specimens.

| | | Standard test(PCR) | | |
|---|---|---|---|---|
| | | Positive | Negative | Total |
| gold nanoparticle | Positive | 19 | 0 | 19 |
| Immunochromatography | Negative | 11 | 60 | 71 |
| Total | | 30 | 60 | 90 |

| Diagnostic sensitivity | 63.3% (19/30) (95% confidence | | |
|---|---|---|---|
| diagnostic specificity | 100.0%(60/60) (95% confidence | | |
| | interval: 96.6%~100%) | | |
| Good Ag COVID-19 | | | |
| Antigen Test | Comparator Method (Standard QTM) | | |
| | Positive | Negative | Total |
| Positive | 19 | 8 | 27 |
| Negative | 0 | 63 | 63 |
| Total | 19 | 71 | 90 |

| | | |
|---|---|---|
| Positive Percent Agreement | 100% | (95% CI: 83.2% |
| (PPA): | | to 100%) |
| Negative Percent Agreement | 88.7% | (95% CI: 79.3% |
| (NPA): | | to 94.2%) |
| Overall Percent Agreement | 91.1% | (95% CI: 83.4% |
| (OPA): | | to 95.4) |

Good Ag test had a sensitivity of 90% and specificity of 100% while competitor assay showed sensitivity of 63.3% and a specificity of 100% and the PPa and NPA of these assays were 100% and 88.7%, respectively. The overall % agreement was 91.1%, which is deemed acceptable.

### [Example 7-2] Clinical Sample Real Time RT-PCR and Comparison Result of 3 Companies Using 20 Positive Samples

There were 20 remnant PCR-positive (as diagnosed by SARS-COV-2 Real-Time RT PCR that got emergency approval from the Korean FDA) nasopharyngeal samples carried in viral transport media from patients suspected of Coronavirus Disease 2019 (SARS-COV-2). The following specifications were considered: date of symptom, sample collection date, confirmed date of diagnosis and preserved at −80° C. after inactivation at 65° C. in 90 μL of buffer for more than 30 minutes. The specifications were used to get diagnostic sensitivity and specificity of the systems and methods herein.

The diagnostic Kit for SARS-COV-2 infection using Latex Beads and Biotin-Polystreptavidin (CL-B/PS LFA) and FDA EUA-approved rapid antigen test from a competitor company (BD biosensor, Seoul, South Korea) using gold nanoparticles were carried out in parallel as a comparison test. The comparison test involved: 10 μL of clinical samples that were preserved at −80° C. after inactivation at 65° C. in 90 μL of buffer for more than 30 minutes were mixed with standard extraction buffer, applied to sample well in a total amount of 100 ul, and test results were interpreted by 2 separate experimenter/interpreters according to provided score card. The FDA EUA-approved rapid antigen test from a competitor company using gold nanoparticles used 10 μL of clinical samples mixed with sample buffer 40 ul and 50 ul was applied in the sample well and reacted for 30 minutes. Visual inspection was then carried out by the same 2 separate experimenter/interpreters.

| | | | | Comparison | | |
|---|---|---|---|---|---|---|
| Sample | Real Time PCR | | | Precision | | SD |
| no | RdRP | ORFlab | N | Bio | Humasis | Biosensor |
| HMN714 | 12.56 | 13.59 | 11.45 | + | + | + |
| HMN715 | 11.38 | 12.48 | 10.83 | + | + | + |
| HMN726 | 20.28 | 21.86 | 20.52 | + | + | + |
| HMN396 | 19.01 | 20.72 | 18.86 | − | − | − |
| HMN404 | 17.56 | 18.83 | 15.12 | + | − | − |
| HMN816 | 20.52 | 22.87 | 2626 | + | + | + |
| HMN879 | 24.83 | 26.74 | 25.91 | + | + | + |
| HMN893 | 14 | 15.38 | 14.64 | + | + | + |
| HMN899 | 12.1 | 12.87 | 11.5 | + | + | + |
| HMN901 | 18.36 | 19.94 | 15.87 | + | − | − |
| HMN907 | 1475 | 1637 | 14.35 | + | + | + |
| HMN910 | 21.74 | 23.08 | 21.69 | + | + | + |
| HMN916 | 16.42 | 18.1 | 17.7 | + | + | + |
| HMN917 | 15.94 | 17.89 | 23.11 | + | + | + |
| HMN928 | 16.35 | 17.97 | 17.93 | + | + | + |
| HMN989 | 2225 | 23.65 | 2333 | + | + | + |
| HMN996 | 23.75 | 25.52 | 26.5 | + | + | + |
| HMN002 | 18.74 | 20.63 | 21.73 | + | + | + |
| HMN003 | 25.56 | 27.23 | 27.23 | + | + | + |
| | | | Positive/20 | 19 | 17 | 17 |
| | | | Sensitivity | 95% | 85% | 85% |
| | | | 95% CI | 75.13% to | 62.11% to | 62.11% to |
| | | | | 99.87% | 96.79% | 96.79% |
| | | | Specificity | 100% | 100% | 100% |

### Conclusion

Of the 20 positive samples, Precision Bio Kit showed 19 positive samples, SD products showed 176 positive samples, and Humasis products showed 17 positive samples. Of the 40 negative samples, Good Ag kit showed 40 negative samples, SD kit showed 40 negative samples, and Humasis kit showed 40 negative samples. The sensitivity of Precision Bio kit in comparison with SD and Humasis was superior (95% versus 85% and 85%).

Comparison test of Diagnostic Kit for SARS-COV-2 infection using Latex Beads and Biotin-Polystreptavidin with FDA EUA-approved rapid antigen test from 2 competitor companies using gold nanoparticle showed the superior sensitivity of the systems and methods herein in diagnosing SARS-COV-2 infection and it can be concurred that the systems and methods herein had a superior sensitivity and compatible specificity of the CL-B/PS LFA method compared to the standard assay using gold nanoparticles.

### Example 8—Standard Operating Procedure for Antigen Test (Good Ag COVID-19 Ag Test Kit)

### Explanation of the Test

Good Ag COVID-19 Ag test Kit (GG CAG-01/02/03) of the systems and methods herein enabled the easy development of customized sandwich lateral flow assays, by combining Lightning-Link® and Latex bead conjugation technologies with an immunochromatography test performed on Universal-LFA strips. The signal intensities have been qualitatively analyzed using the supplied scoring card or, for a quantitative detection, an LFA reader can be used.

The “Good Plus COVID-19 antigen test” included a lateral flow assay, which can: (i) detect as little as 0.01 ng/ml N (Nucleoprotein) antigen of SARS-Cov-2 in nasal (GG HAG-01/02; home use)/nasopharyngeal or oropharyngeal or nasal swab (GG CAG-01/02/03; professional use) specimens with higher analytical sensitivity than most if not all the commercially available antigen kits (LOD 10-20 time lower); (ii) shows higher clinical performance (sensitivity 85-95% and specificity 100% in reference to real time PCR assay) that allows ordinary people to detect SARS-Cov-2 within 20 minutes without need of additional instrumentation.

The Good Plus COVID-19 Ag test was a rapid, qualitative immunochromatographic assay for the presence of SARS-COV-2 antigens in human nasopharyngeal swab specimens. The test line in each device contained mouse monoclonal antibodies to the nucleoprotein (NP) of SARS-CoV-2. When the sample contained SARS-COV-2 antigens, anti-SARS-COV-2 monoclonal antibodies coupled with biotin in the biotin pad, SARS-COV-2 became bound to antigens in the sample to form an antigen-antibody complex. This complex moved by capillary action to the latex conjugation pad where it formed a biotin-antigen-antibody complex. This was later captured by anti-SARS-COV-2 monoclonal antibodies coupled with polystreptavidin immobilized on the Test line (test zone) by the biotin-polystreptavidin bond, and a visible line appears on the membrane, while unbound dye complexes continued to migrate beyond the test line area by capillary action. When the sample was adequate, unbound protein-dye complexes were coupled with the anti-chicken IgY antibody and the colloidal gold and show up captured at the Control line (control zone) as a red line. Formation of the Control line served as an internal control. If the Control line did not appear within the designated incubation time (i.e., 20 minutes), the result was invalid and the test should be repeated with a new sample.

The capture antibody was conjugated to Biotin, and the detection antibody was conjugated to 400 nm Latex bead with the Latex bead Conjugation Kit of the systems and methods herein, both of which require only 30 seconds to set up.

The capture and detection antibodies were diluted and incubated with the analyte and then run on Universal LFA strips of the systems and methods herein. Universal LFA strips consisted of: a nitrocellulose membrane containing a ‘Test line’ (T-line) of immobilized SARS-COV-2 NP monoclonal antibody, which: (i) binds the Biotin conjugated capture antibody and (ii) further binds the analyte in complex with the Latex bead-detection antibody. T-line exploited the extraordinarily high affinity of streptavidin for biotin and a red T-line appears when the analyte is present and the line intensity varies, depending on the analyte concentration. Good Plus COVID-19 antigen test LFA strips of the systems and methods herein also contained: (i) a ‘Control-line’ (C-line), which shows that the test is valid, and (ii) an absorbent pad, which promotes and controls the flow of sample through the membrane.

### Principles of the Test

The Good Ag COVID-19 Antigen Test is a manually performed, visually read immunoassay for the qualitative detection of SARS-COV-2 nucleocapsid protein antigen using a proprietary integrated collection swab to directly collect samples from the anterior nasal cavity. The Good Ag COVID-19 Antigen Test is comprised of both a single-use test device and a vial containing a pre-measured amount of a buffered Extraction buffer solution. The test consists of a sealed pouch with two separate compartments for each component. The Good Ag COVID-19 Antigen Test utilizes a proprietary lateral flow immunoassay procedure.

The assay test strip, which can be viewed through the test device result window, is comprised of a series of components: the blocker pad, the conjugate pad, the nitrocellulose membrane, and finally the absorbent pad. The performance of the assay occurs by hydration and transport of reagents and specimen as they interact across the strip via chromatographic lateral flow.

An anterior nasal sample is collected using the flat pad that is integrated into the test device, followed by swirling the test device in the vial of Extraction buffer solution. The Extraction buffer solution facilitates the flow of the sample into the device and onto the test strip. As the sample flows through the device, it rehydrates the reagents on the blocker pad, which contains biotinylated anti-SARS-COV-2 antibodies. The sample then re-hydrates the gold colorimetric reagent, which contains anti-SARS-COV-2 antibodies. If the sample contains SARS-COV-2 nucleocapsid protein antigen, it will react with the anti-SARS-CoV-2 antibodies in the blocker pad and conjugate pad and forms a sandwich complex that migrates up the test strip. As the complex continues to migrate up the test strip it encounters the Test (T) Zone and will react with the streptavidin immobilized on the nitrocellulose, a reddish-purple line will appear, qualitatively indicating the presence of SARS-COV-2 nucleocapsid antigen in the sample. The intensity of the line color is not directly proportional to the amount of antigen present in the sample. If the sample does not contain SARS-COV-2 nucleocapsid protein antigen, the sandwich complex will not form and the reagents will flow past the Test (T) Zone.

Further up the test strip, the sample will encounter the Control (C) Zone. This is a built-in procedural control which serves to demonstrate that the fluid migrated through the test device. For negative results and most positive results a line will form at the Control (C) Zone. In some cases when viral levels are high, the line at the Control Zone may be very faint or may not be present.

Results are interpreted between 20 and 30 minutes after inserting the device into the Extraction Tube. Do not read negative results before 30 minutes as it may result in false negative results. Do not read any result after 30 minutes as it may result in inaccurate results.
1. Collect samples as soon as possible within 5 days of symptom onset.
2. The sample should be treated with lysis buffer as soon as possible after collection. The processed sample in buffer vial may be stored at 2 to 8° C. for 2 days, or at −20° C. for 3 months, or at −70° C. for long term storage. However, storage after 1 hour has not been verified and each storage condition for any particular environment must be determined by additional experimentation.
3. If the sample cannot be immediately disposed, the sample should be put into the buffer above and tightly sealed for storage, usually at 2 to 8° C. for 1 day, or −70° C. for long term storage. Avoid freezing-thawing repeatedly.

### Materials Provided

Good Ag COVID-19 Antigen Test Kit is available in the following packaging configuration:
Components of Kit Catalog Number
GG-CAG-01 Unit Box (1 pack)
Unit Box (1pack)
GG-CAG-02 Unit Box (1 pack)
GG-CAG-03

### Unit Box Containing:

Pouch Containing: Test Device (1/Test)
Nozzle Cap (1/Test) Extraction Tube (1/Test)
(each vial contains 0.40 mL of a extraction buffer with an antimicrobial agent)
Test Stand (1/Test)

### Instructions for Use

Sterile Nasopharyngeal swabs
1 Quick User Guide (in English and Korean) 10 Test/kit 25 Test/kit 50 Test/kit

*Extraction tube: Buffer Considerations:

The buffer should be stored in room temperature. (20-24° C.). However, temperatures between 0 to 30° C. (32 to 86° F.) does not affect test function. Keep kit in a parallel surface once the seal is open as spilling of the buffer may effect test results.

Individually the concentrations shown should not affect the reaction. However, in combination with additional compounds that are not recommended above a certain concentration, the reaction may be affected.

The lysis buffer is used for rapid extraction of virus antigen from swab samples.

### Materials Required but not Provided

Timer or watch capable of timing

### Warnings and Precautions

Test devices that contain patient samples should be handled as though they could transmit disease. Follow universal precautions1 when handling samples, this kit, and its contents. Wear appropriate personal protection equipment (PPE)2 and gloves when running the test and handling a patient's test device. Change gloves between tests.

This test is for the detection of SARS-COV-2 antigen, not for any other viruses or pathogens

Laboratories within the United States and its territories are required to report all results to the appropriate public health agencies.

Do not use test kit if it is past the expiration date.

Follow the Instructions for Use to obtain accurate results. Incorrect sampling may result in false results.

False Negative results can occur if negative results are read before 30 minutes.

Invalid results can occur if the swab is not stirred at least 10 times.

If any of the solution in the Extraction Tube spills, it may cause invalid results. You need to repeat testing with a new test.

### Device Handling Precautions

Do not reuse the Test Device and Extraction Tube.

Inspect the Divided Pouch. If the Divided Pouch has been damaged, discard the Divided Pouch and its contents and select a new Divided Pouch for testing.

Do not interchange Test Devices and Extraction Tubes from kits with different lot numbers.

If the Test Device is not immediately inserted into the Extraction buffer Solution after sample collection, remove the Nozzle Cap from the Divided Pouch and place the Test Device into the Divided Pouch for transport or until the device can be inserted into the Extraction buffer Solution. The Test Device must be inserted into the Extraction buffer Solution within 30 minutes of collection.

Adequate lighting is required to read a test result.

The solution in the tube contains potentially harmful chemicals (Triton X-100 and ProClin 950); however, laboratory studies have shown them to be nontoxic at the levels contained in the solution. The Extraction buffer solution should only be used as directed; do not ingest; keep out of the reach of children; avoid contact with skin and eyes. If the solution contacts the skin or eye, flush with copious amounts of water. If irritation persists, seek medical advice: https://www.poison.org/contact-us or 1-800-222-1222.

### Storage Instructions

Store unused Good Ag COVID-19 Antigen Test kits unopened at 2°-30° C. (35°-86° F.). Do not open the Divided Pouch until you are ready to perform the test. If stored refrigerated, ensure that the Divided Pouch is brought to operating temperature (15°-40° C., 59°-104° F.) before opening.

### Quality Control Procedures

### Built-In Control Features

The Good Ag COVID-19 Antigen Test for anterior nasal specimens has a built-in procedural control that demonstrates the assay components have migrated adequately through the device. For negative tests, a reddish-purple line in the Control (C) Zone of the Result Window indicates that the fluid migrated appropriately through the Test Device. The line in the Control (C) Zone does not determine if a human sample has been added or if there is an adequate sample. For most positive tests, a reddish-purple line will appear in the Control

(C) Zone and the Test (T) Zone; however, in cases where the viral load in the sample is very high, the line in the Control (C) Zone may not be present or may be very faint. (Refer to Test Result and Interpretation of Test Result section in these Instructions for Use).

### Instructions for Use

Follow Safety Precautions section in these Instructions for Use.

Gather all the materials you will need. Allow the Good Ag COVID-19 Antigen Test to come to operating temperature (15°-40° C., 59°-104° F.) before use. Personnel collecting specimens or working within 6 feet of patients suspected to be infected with SARS-COV-2 should maintain proper infection control and use recommended personal protective equipment (PPE), which could include an N95 or higher-level respirator (or face mask if a respirator is not available), eye protection, gloves, and a lab coat or gown.

It is recommended that personnel wear well-fitting cloth masks, facemasks, or respirators at all times while at the point-of-care site where the testing is being performed.

### Specimen Collection and Test Procedure

Set the Test Stand at your workspace. Make sure the Test Stand is on a sturdy surface. Use only the Test Stand provided.
1. Your box may contain more than one test kit. Use only 1 of each of the following for each test:
2. Open the pouch just prior to use, lay it flat and perform assay as follows (see picture 1).
3. Remove the Extraction Tube (“Vial”) from the Pouch
4. Slide the Vial upright into the top of one of the slots in the Test Stand. DO NOT force the Vial into the Stand from the front of the slot as splashing may occur. Make sure the Vial is pushed all the way to the bottom of the slot in the Test Stand. If solution spills out of the vial, you will need to obtain a new test. (see picture 2).
   Keep vial upright so that it does not spill
5. Remove the Device from its Pouch (see picture 3).
6. Remove one of the sterile swabs from its cover from its stick end, being careful not to touch the tip.
   (see picture 4).
7. Collect Nasal Swab (Proceed to alternate procedure below if nasopharyngeal swab is needed or self collected swab is used)
   Insert the entire absorbent tip of the swab (usually ½ to ¾ of an inch) into one nostril. Slowly remove the swab
   Firmly brush against insides of nostril in a circular motion 5 times or more for at least 15 seconds. Do not just spin the swab.
   Using the same swab, repeat sample collection in the other nostril (see picture 5).
   Check:
   Did you swab BOTH nostrils?
8. Locate the extraction vial and gently peel off the aluminum foil seal, being sure to keep the vial upright and place it in the packaging tray (see picture 6).
9. Place the swab into the extraction vial. Rotate the swab vigorously at least 5-10 times in rotation for about 10 seconds. At the same time, press the swab head against the wall of the tube to release the antigen (see picture 7).
10. To drain the liquid from the swab as much as possible, remove the swab by rotating against the extraction vial while squeezing the sides of the vial to release the liquid from the swab. Discard the swab as per biohazard waste disposal method.
11. Close the vial with the provided cap and =push the cap firmly onto the vial. Mix thoroughly by flicking the bottom of the tube. Press the nozzle cap tightly onto the tube. (see picture 8).
12 Apply 3 drops of extracted specimen to the specimen well of the test device. (see picture 9).
13. Make sure the tube and device are on a flat surface.
14. Start timing the test (see picture 10) by setting the timer for 20 minutes. DO NOT remove the Device from the Vial while the test is running.
   Note: A control line may appear in the result window in a few minutes but a sample line may take as long as 15-20 minutes to appear.
   Note: Results should not be read after 30 minutes.
15. Pink fluid will appear and travel up the Result Window. The pink fluid will gradually disappear as the test develops (see picture 11).
16. If you don't have symptoms, a second test should be taken at least 24 hours (and no more than 48 hours) after the first test.

### Test Result and Interpretation of Test Result

Interpret results between 20 and 30 minutes. Do not read negative results before 30 minutes as it may result in false negative results. Do not read any result after 30 minutes as it may yield inaccurate results.

### Negative

A test is Negative if:

A reddish-purple line appears in the C Zone and NO line appears in the T Zone (see picture 12). The line in the C Zone must be present to interpret a negative test result.

A Negative test result is interpreted as nucleocapsid protein antigen was not detected in the specimen. The individual is presumed negative for COVID-19.

Negative results do not rule out SARS-CoV-2 infection. Individuals without symptoms that test negative should be tested again with at least 24 hours and no more than 48 hours between tests. All negative results are considered presumptive, and confirmation with a molecular assay, if necessary for patient management, may be performed. Negative results should be considered in the context of an individual's recent exposures, history, and the presence of clinical signs and symptoms consistent with COVID-19.

### Positive

A test is Positive if:

A reddish-purple line appears in the T Zone and there is a line in the C Zone. Lines may vary in intensity. The test is positive regardless of how faint these lines appear (see picture 13).

In some cases the reddish-purple line in the C Zone may not be present or may be very faint if there are high levels of virus in the sample (see picture 14).
Look very closely!
The bottom line can be very faint.
Any pink/purple line visible at test line is a Positive Result.

A Positive test result is interpreted as nucleocapsid protein antigen was detected in the specimen. The individual is positive for COVID-19. Additional confirmatory testing with a molecular test for positive results may also be necessary, if there is a low likelihood of COVID-19, such as in individuals without known exposures to COVID-19 or residing in communities with low prevalence of infection.

### Invalid

A test is Invalid if any of the following occurs:
NO lines appear on the device (see picture 15), or
a reddish-purple background in the Result Window makes it difficult to read the result after 30 minutes (see picture 16), or
any partial line on one side of the C or T Zones (see pictures 17 and 18)

An Invalid test result means that there was a problem running the test. An Invalid result cannot be interpreted. An invalid test result needs to be repeated with a fresh sample and a new test device. Please contact Cellgenemedix Customer Care (1-832-468-6985) if you are unable to obtain a valid test result upon repeat testing.

### Specimen Collection and Test Procedure

Set the Test Stand at your workspace. Make sure the Test Stand is on a sturdy surface. Use only the Test Stand provided.
17. Open the pouch just prior to use, lay it flat and perform assay as follows (see picture 1).
18 Remove the Extraction Tube (“Vial”) from the Pouch.
19 Slide the Vial upright into the top of one of the slots in the Test Stand. DO NOT force the Vial into the Stand from the front of the slot as splashing may occur. Make sure the Vial is pushed all the way to the bottom of the slot in the Test Stand. If solution spills out of the vial, you will need to obtain a new test.
20 Have the patient blow his/her nose into a tissue. DO NOT clean out nose with the tissue.
21. Remove the Device from its Pouch (see picture).
22. Remove one of the sterile swabs from its cover
   (see picture).
23. Professional Nasal Swab (Proceed to alternate procedure below if nasopharyngeal swab is needed or self collected swab is used)
   Place the Insert the nasal swab into the nostril exhibiting the most drainage or congestion
   Using gentle rotation, push the swab until resistance is met at the level of the nasal turbinates (Less than one inch into nostril)
   Rotate the swab 5 times or more against the nasal wall
   Slowly remove the swab
   Using the same swab, repeat sample collection in the other nostril.
24. Locate the extraction vial and gently peel off the aluminum foil seal, being sure to keep the vial upright and place it in the packaging tray.
25. Place the swab into the extraction vial. Rotate the swab vigorously at least 5-10 times in rotation for about 10 seconds. At the same time, press the swab head against the wall of the tube to release the antigen.
26. To drain the liquid from the swab as much as possible, remove the swab by rotating against the extraction vial while squeezing the sides of the vial to release the liquid from the swab. Discard the swab as per biohazard waste disposal method.
27. Close the vial with the provided cap and push the cap firmly onto the vial. Mix thoroughly by flicking the bottom of the tube. Press the nozzle cap tightly onto the tube.
28. Apply 3 drops of extracted specimen to the specimen well of the test device.
29. Make sure the tube and device are on a flat surface.
30 Start timing the test (see picture 13) by setting the timer for 20 minutes. DO NOT remove the Device from the Vial while the test is running.
31. Pink fluid will appear and travel up the Result Window. The pink fluid will gradually disappear as the test develops (see picture 14).

### (Alternate Procedures for 9.)

### Oropharyngeal (OP) Swab

(recommended in patients with predominantly upper respiratory symptoms)
1. Insert swab into the posterior pharynx and tonsillar areas.
2. Rub swab over both tonsillar pillars and posterior oropharynx and avoid touching the tongue, teeth, and gums.
3. Place swab, tip first, into the transport tube provided.

*Both NP and OP specimen are acceptable specimen types. If both NP and OP specimens are collected, combine them in a single tube to maximize test sensitivity and limit use of testing resources.

### Self Collected Nasal Swab:

1. Tilting the head back slightly, place the nasal swab into one nostril, insert the swab about 1 inch into the nasal cavity. Take care that the swab is parallel to the roof of mouth, not upwords. The entire swab tip should be in the nostril.
2. Firmly pressing the swab against the nasal wall, rotate the swab 15 times.
3. Do not swab both nostrils unless otherwise instructed by your healthcare provider (see pictures 10). If you do not swab both nostrils 15 times each, you may get a false result.

### Test Result and Interpretation of Test Result

Interpret results between 20 and 30 minutes. Do not read negative results before 30 minutes as it may result in false negative results. Do not read any result after 30 minutes as it may yield inaccurate results.

### Negative

A test is Negative if:

A reddish-purple line appears in the C Zone and NO line appears in the T Zone (see picture 15). The line in the C Zone must be present to interpret a negative test result.

A Negative test result is interpreted as nucleocapsid protein antigen was not detected in the specimen. The individual is presumed negative for COVID-19.

Negative results do not rule out SARS-COV-2 infection. Individuals without symptoms that test negative should be tested again with at least 24 hours and no more than 48 hours between tests. All negative results are considered presumptive, and confirmation with a molecular assay, if necessary for patient management, may be performed. Negative results should be considered in the context of an individual's recent exposures, history, and the presence of clinical signs and symptoms consistent with COVID-19.

### Positive

A test is Positive if:

A reddish-purple line appears in the T Zone and there is a line in the C Zone. Lines may vary in intensity. The test is positive regardless of how faint these lines appear (see pictures 16 and 17).

In some cases the reddish-purple line in the C Zone may not be present or may be very faint if there are high levels of virus in the sample (see picture 18).

A Positive test result is interpreted as nucleocapsid protein antigen was detected in the specimen. The individual is positive for COVID-19. Additional confirmatory testing with a molecular test for positive results may also be necessary, if there is a low likelihood of COVID-19, such as in individuals without known exposures to COVID-19 or residing in communities with low prevalence of infection.

### Invalid

A test is Invalid if any of the following occurs:

NO lines appear on the device (see picture 19), or a reddish-purple background in the Result Window makes it difficult to read the result after 30 minutes (see picture 20), or any partial line on one side of the C or T Zones (see pictures 21 and 22)

An Invalid test result means that there was a problem running the test. An Invalid result cannot be interpreted. An invalid test result needs to be repeated with a fresh sample and a new test device. Please contact

Cellgenemedix Customer Care (1-832-468-6985) if you are unable to obtain a valid test result upon repeat testing.

### Reporting Results

All healthcare providers are required to report all test results from individuals who use the authorized product to relevant public health authorities in accordance with local, state, and federal requirements using appropriate codes, as defined by the health department.

### General Test Clean-Up

1. Dispose of the used test materials in a biohazard waste container. All equipment and biohazardous waste should be discarded in accordance with country, state, and local laws and policies.
2. Change your gloves between each test to prevent contamination.
3. Use a freshly prepared 10% solution of bleach to clean up any spills.

### Limitations of the Test

1. Testing for asymptomatic individuals should be performed at least twice over three days with at least 24 hours and no more than 48 hours between tests.
2 There is a higher chance of false negative results with antigen tests than with laboratory-based molecular tests. This means that there is a higher chance this test will give a negative result when the patient has COVID-19.
3. Serial testing (i.e., testing every day or every other day) is more likely to detect COVID-19, especially when the patient does not have any symptoms.
4. A negative test result may occur if the level of antigen in a sample is below the limit of detection of the test.
5. Weak Positive samples may take longer to develop and can take the entire 30 minutes for a test line to be present. Therefore, all negative test results must be read at least 30 minutes after inserting the device into the Extraction Tube. Negative test results must not be reported prior to reading the device at 30 minutes.
6. Reading any result after 30 minutes may yield inaccurate test results.
7. The control line only indicates that reagents have properly migrated up the test device. In positive patient samples with high levels of virus, the line at the Control (C) Zone may not be present or may be very faint. The control line does not indicate that an adequate human sample was added to the test device.
8. Positive test results do not rule out co-infections with other pathogens.
9. Potential cross reactivity of the Good Ag COVID-19 Antigen Test with COVID-19 vaccines or therapeutics has not been evaluated.
10. False negative results may occur if a specimen is improperly collected or handled.
11 False negative results are more likely after seven days or more of symptoms.
12 Negative results are presumptive, do not rule out COVID-19 infection and it may be necessary to obtain additional testing with a molecular assay, if needed for patient management.
13. Performance of nasal swabs collected from patients without symptoms or other epidemiological reasons to suspect COVID-19 infection or for serial screening, when tested twice over three days with at least 24 but not more than 48 hours between tests has not been determined. A study to support use will be completed.
14 If the differentiation of specific SARS viruses and strains is needed, additional testing, in consultation with state or local public health departments, is required.
15. The performance of this test was established based on the evaluation of a limited number of clinical specimens collected in February and April 2021. The clinical performance has not been established in all circulating variants but is anticipated to be reflective of the prevalent variants in circulation at the time and location of the clinical evaluation. Performance at the time of testing may vary depending on the variants circulating, including newly emerging strains of SARS-COV-2 and their prevalence, which change over time.

### Conditions of Authorization for Healthcare Providers

To assist Healthcare Providers prescribing or using the Good Ag COVID-19 Antigen Test, the relevant Conditions of Authorization are listed below: All healthcare providers are required to report all test results from individuals who use the authorized product to relevant public health authorities in accordance with local, state, and federal requirements using appropriate codes, as defined by the health department.

Correct practices when performing tests in point-of-care settings:
1. Before the Test
   Perform a risk assessment to identify what could go wrong, such as breathing in infectious material or touching contaminated objects and surfaces. Then
   Implement appropriate control measures to prevent these potentially negative outcomes from happening.
   Use a new pair of gloves each time a specimen is collected from a different person. If specimens are tested in batches, also change gloves before putting a new specimen into a testing device. Doing so will help to avoid cross-contamination.
   Do not reuse used test devices, reagent tubes, solutions, swabs, lancets, or fingerstick collection devices.
   Store reagents, specimens, kit contents, and test devices according to the manufacturer's instructions found in the package insert.
   Discard tests and test components that have exceeded the expiration date or show signs of damage or discoloration (such as reagents showing any signs of alteration).
   Do not open reagents, test devices, and cassettes until the test process is about to occur. Refer to the manufacturer's instructions to see how long a reagent, test device, or cassette can be used after opening.
   Label each specimen with appropriate information to definitively connect that specimen to the correct person being tested.
   When transferring specimens from a collection area to a testing area, follow the instructions for the point-of-care test used.
2. During the Test.
   Follow all the manufacturer's instructions for performing the test in the exact order specified.
   Perform regular quality control and instrument calibration, as applicable, according to the manufacturer's instructions. If quality control or calibration fails, identify and correct issues before proceeding with patient testing.
   When processing multiple specimens successively in batches, ensure proper timing for each specimen and each step of the testing process, as specified by the test manufacturer. To avoid cross-contamination, change gloves before putting a new specimen into a testing device.
3. After the Test.
   Read and record results only within the amount of time specified in the manufacturer's instructions. Do not record results from tests that have not been read within the manufacturer's specified timeframe.
   Decontaminate the instrument after each use. Follow the manufacturer's recommendations for using an approved disinfectant, including proper dilution, contact time, and safe handling.
   Always discuss used and unused COVID-19 test kit waste with your facility leadership, facility waste management contractor, your State Department of Public Health, and the test manufacturer's technical support. All waste disposal must comply with your local, tribal, regional, state, national, and/or international regulations.

### Quality Control

Internal procedural controls are included in the test. A red line appearing in the control line region (C) is an internal procedural control. The appearance of the procedural control line indicates that proper volume of specimen has been added and capillary flow occurred. If the procedural control line does not develop in 20 minutes, the test result is considered invalid, and retesting with a new cassette is recommended.

### 8.4 Buffer Considerations:

The buffer of the kit of the systems and methods herein were stored in room temperature. (20-24° C.). However, temperatures of 0-40° C. does not affect test function. The kit was kept in a parallel surface once the seal is open as spilling of the buffer may impact test results. Individually, the concentrations shown should not affect the reaction. However, in combination with additional compounds that were not recommended above a certain concentration, the reaction may be impacted. The lysis buffer was used for rapid extraction of virus antigen from swab samples.

TABLE 11
Recommended Starting Amount and Volume of sample:

| | Maximum Amount | Fixed Volume |
|---|---|---|
| Vial size | of specimen | of buffer |
| 4 mL | 200 μL | 400 μL |

Superior reactions (i.e., reactions leading to accurate and reproducible results) were normally generated using ˜100 μL of specimen. Specimen with antigen concentration less than 1 mM can still be used to generate good results provided the maximum conjugation volume is not exceeded. Note that adding less than the required amount of specimen may result in unbound label post conjugation (hook effect).

Your Good Ag COVID-19 Ag kit can be stored at RT (room temperature) for up to 12 months. For longer storage, the Good Ag COVID-19 kit can be stored at 4° C. The best storage conditions for any particular environment must be determined by additional experimentation.

While the Good Ag COVID-19 test Kit allows point of care testing (POC), it is preferred that GG-CAG-03 with nasopharyngeal swabs, the sample collection be done by a health care provider.

The hands-on time for the sample acquisition and buffer mix procedure was about 1-2 minutes and the Good Ag COVID-19 test was ready to interpret within 20 minutes.

TABLE 12
Buffer Components

| Buffer components* | |
|---|---|
| pH | 8.6-9.0 |
| Amine free buffer (e.g. MES, MOPS, HEPES) | O |
| Glycerol | None |
| Tris | 100 mM |

Not hazardous waste (Hazardous Components: None in reportable quantities). Refer to MSDS in https://cellgenemedix.com/msds/
*0.1M Tris-HCl, 0.5% Casein from Bovine milk, 0.5% Tween 20, 1% IGEPAL CA-630, 0.5% EDTA, 0.1% Sodium Azide

### 8.5—Actual Preparation and Implementation of Kit of the Systems and Methods Herein

1. Wash hands thoroughly for at least 20 seconds before the preparing test kit of the systems and methods herein.
2. Unpack the components of the kit of the systems and methods herein from the tray.
3. Remove from the device pouch and the test device place the device on a flat, clean surface.
4. Locate the extraction vial and gently peel off the aluminum foil seal, being sure to keep the vial upright and place it in the packaging tray.
5. Locate a nasal swab and remove from the pouch. Be careful not to touch the swab tip.

### Nasal Swab Collection (G-CHAG-01)

1. Gently insert the swab ¾ inch (about 2 cm) or until resistance is felt into the LEFT nostril. Then, slowly rotate the swab at least 5 times in a circular path for a total of 15 seconds.
2. Gently remove the swab from the LEFT nostril and place directly into the RIGHT nostril, repeating the process of rotating at least 5 times in a circular path for a total of 15 seconds. Remove the swab from the RIGHT nostril.
3. Slowly remove the swab from the nostril while rotating it. Be careful not to touch the swab tip.
NOTE: The right nostril may be sampled first as long as both nostrils are sampled.

### Extraction Vial:

1. Locate the extraction vial and gently peel off the aluminum foil seal, being sure to keep the vial upright and place it in the packaging tray.
2. Place the swab into the extraction vial. Rotate the swab vigorously at least 10 times in rotation for about 10 seconds. At the same time, press the swab head against the wall of the tube to release the antigen.
3. To drain the liquid from the swab as much as possible, remove the swab by rotating against the extraction vial while squeezing the sides of the vial to release the liquid from the swab. Discard the swab as per biohazard waste disposal method.
4. Close the vial with the provided cap and push the cap firmly onto the vial. Mix thoroughly by flicking the bottom of the tube.

### LFA Device:

1. Invert the extraction vial and hold the sample vertically above the sample well. Squeeze the vial gently. Allow THREE (3) drops of sample to fall into the sample well. Leakage of the sample is possible when 5 drops or more of the sample are added.

### LFA Device Application and Interpretation:

1. Start the timer. Read the result at 15 minutes.
2. The test result should not be read after 20 minutes and DO NOT move or lift the test device during this time. A qualitative readout was cost-effective, requiring only a visual assessment, and for which there was a score card. A quantitative readout was more expensive, requiring an LFA strip reader. An LFA strip reader allowed the creation of a calibration curve and thereby providing reproducible and accurate measurements.
3. Positive Result was when two distinct colored lines appear (e.g., one red-colored line next to “C” and one red-colored line next to “T”, indicating a COVID-19 positive result). NOTE: The color intensity in the test region varied depending on the amount of SARS-COV-2 nucleocapsid protein antigen present in the sample. Any faint colored line(s) in the test region(s) should be considered as positive. If there was a positive test result, it was very likely that the patient had COVID-19 because proteins from the virus that causes COVID-19 were found in the sample. Therefore, the patient was may be placed in isolation to avoid spreading the virus to others.

There was a very small chance that this test can give a positive result that is wrong (a false positive result). If a patient tested positive with the Good Ag COVID-19 test kit, the patient isolated and sought follow-up care, as additional testing may be necessary. Your healthcare provider as additional testing may be necessary. The healthcare provider can work with the patient to determine how best to care for you based on your test result(s) along with your medical history, and your symptoms.
4. Negative Result was when only one red-colored line was next to “C”, indicating a negative result. A negative test result means that proteins from the virus that causes COVID-19 was not found in your sample. It was possible for this test to give a negative result that is incorrect (false negative) in some people with COVID-19. This means you could possibly still have COVID-19 even though the test is negative.

The amount of antigen in a sample may decrease the longer you have symptoms of infection. In symptomatic people, specimens collected after you have had symptoms for more than five days may be more likely to be negative compared to a molecular assay.

If you test negative and continue to experience COVID-19 like symptoms of fever, cough and/or shortness of breath you should seek follow up care with your healthcare provider. For example, your healthcare provider may suggest you need another test to determine if you have contracted the virus causing COVID-19. If you are concerned about your COVID-19 infection status after testing or think you may need follow up testing, please contact your healthcare provider.
5. Invalid Result was when the red-colored line in the control region “C” was not visible, indicating an invalid result. For an invalid result, the test was re-run one time using the remaining specimen in the extraction vial.
6. Dispose of all used test kit components and swab samples in household trash.

For accurate results, avoid the following: operating the test kit outside of storage conditions; using on anyone under 2 years of age; use within the reach of children; interpreting the test result before 15 minutes and after 20 minutes after starting the test; use on anyone who is prone to nosebleeds or has had facial or head injury/surgery in the last 6 months; use if the test device package is damaged; touching the tip (specimen collection area) of the swab; using the kit contents beyond the expiration date; eating, drinking, or smoking in the area where the specimens and kit contents are handled; interchanging kit contents from different lots; and re-using any contents in the kit as they are single-use only.

Note: Reagents do not contain sodium azide. However, extraction solution should not be ingested. Eye and skin contact with the extraction solution should be avoided.

### Example 9—Standard Operating Procedure for Antigen Test (Good Ag COVID-19 Ag Test Kit)

### Production Manual of the Good Plus SARS-COV-2 Omicron Antigen Test Utilizing Latex Beads and Biotin-Polystreptavidin

### 1. Purpose

In this example of invention, Omicron S Antigen and S antibody were used in place of SARS-COV-2 NP antigen and antibody to create a rapid kit that will detect Omicron SARS-COV-2 more accurately. In a variation of this invention, both anti SARS-COV-2 NP antibody and anti-Omicron S antibody may be used in the biotinylated/latex pads and cut in a way so that there are 2 test lines, 1 for detection of SARS-COV-2 and the other for detection of Omicron SARS-COV-2.

2. Scope of application

| Product | In vitro diagnostic medical device | | |
|---|---|---|---|
| group | | | |
| Product | High-risk infectious agent | Item | K05030. |
| name | immunological test reagent | classification | 01 |
| Model | Good Ag COVID-19 Omicron | Class | Class 3 |
| name | antigen test | | |

| | |
|---|---|
| Manufacturer | PRECISION BIO Co., Ltd./Cellgenemedix Corp |
| Country | Republic of Korea |

### 3. Shape and Structure

### 1) Background

This product is for checking the presence or absence of Omicron S antigen in nasal, oropharyngeal, or nasopharyngeal swabs collected from a person with symptoms of recent onset (less than 6 days) of coronavirus infection symptoms through immunochromatography.

### 2) Appearance

### 3) Components and Characteristics of the Product

### (1) Components of the Product

| Components | Appearance Features |
|---|---|
| 1. Device | It is a white rectangular plastic |
| | cassette. There is a circular |
| | sample drop site at the bottom |
| | of both sides, and the positions |
| | of the control line (c) and the |
| | test line (T) are marked on the |
| | display window. |
| 2. Sample extract | It is a colorless and transparent |
| | liquid contained in a colorless |
| | transparent tube. |
| 3. User Manual | Paper print |
| 4. Swap | A sponge is attached to the end |
| | of the plastic material. |
| 5. Nozzle cap | It is a colorless and transparent |
| | plastic material. |

### (2) Characteristics

### Principle

Good Ag COVID-19 Omicron Test has a control line and a test line on the membrane. In this example of invention, Omicron S Antigen and S antibody were used in place of SARS-COV-2 NP antigen and antibody to create a rapid kit that will detect Omicron SARS-COV-2 more accurately. The control line is coated with anti-chicken IgY antibody, and the test line is coated with streptavidin. No line appears on the result window before sample input. The high surfactant in the swab extraction solution elutes the antigenic proteins in the virus in the extraction solution. When the extracted sample is instilled, if SARS-COV-2-Omicron S antigen is present in the sample, it binds to the anti-Omicron S mAb bound to the biotin pad to form an antigen-antibody complex. This complex moves along the membrane to the latex bonding pad through capillary phenomenon to form the antibody biotin-antigen-antibody latex complex. The formed complex moves along the membrane to the test line, binds to the streptavidin fixed to the test line, and displays a red line in the result confirmation window. If the SARS-COV-2 Omicron S antigen is not present in the sample, no red line is displayed on the test line, and the control line is displayed in red if the test procedure is performed correctly.

In a variation of this invention, both anti SARS-COV-2 NP antibody and anti-Omicron S antibody may be used in the biotinylated/latex pads and cut in a way so that there are 2 test lines, 1 for detection of SARS-COV-2 and the other for detection of Omicron SARS-COV-2.

The systems and methods herein are directed to a CL-B/PS LFA for detection of Omicron type SARS-COV-2.

### Manufacturing Details

The ingredients for CL-B/PS LFA-O include the following below.

The Carboxyl Latex Bead was purchased from Magspher Inc (CA, USA); 400 nm-sized Carboxylic red latex beads is 400 nm (CAB400NM, CAB4865B-1220).

The NHS-biotin (20217, VJ309468) was purchased from Thermo Scientific (MA, USA).

The latex Sulfo-NHS (24510, VL308261), as used in this example, provided microsphere conjugation. The latex Suflo-NHS was purchased from Thermo Scientific InC (MA, USA) and EDC (E7750, BCCD7592) from Sigma-Aldrich InC (St. Louis, USA).

The polystereptavidin, as used in this example, was Polystreptavidin R (ABIN4370319, K664-M/250321) and purchased from Antibodies InC (Aschen, Germany).

The standard antigen for SARS-COV-2 included: a S protein Omicron antigen made by recombinant DNA technology from E. coli using recombination technology; and purified (COVID-19 S Rec. Ag. SPN-C5224 SARS-COV-2 Nucleocapsid protein, His Tag (B.1.1.530/Omicron)). This has been purchased from AcroBiosystems, InC (China) and used instead of SARS COV0-2 NP antigen, but not restricted to this.

For primary and secondary antibodies, 2 monoclonal antibodies made for S antigen of Sars-COV; SARS-COV-2 S mAb (Anti-SARS-COV-2 Spike RBD Neutralizing Antibody, Chimeric mAb, Human IgG1 (AM122)); and Anti-SARS-CoV-2 Spike RBD Antibody, Mouse IgG1 (SPD-M305) was used and purchased from AcroBiosystems, InC (China) instead of AP antibodies, but not restricted to this.

The Goat anti-chicken IgY antibodies (20900, CB25203) was used for control line and purchased from Boreda biotech, InC (Seong nam, South Korea), but not restricted to this.

Boric acid (B0394), Casein (C7078), MES hydrate (M2933), EDC (3003026795), Sodium phosphate monobasic (S0751), Sodium phosphate dibasic (S0876), Tween-20 (P2287), BSA (A7906) was purchased from Sigma-Aldrich InC (St. Louis, USA).

The striping of the systems and methods herein included: sample pad, conjugation pad, nitrocellulose membrane), and absorbent pad, with backing card. The nitrocellulose membrane (IAB090-E1, 00221107) was purchased from Advantech InC (Tai-pei, Taiwan), and absorbent pad (Grade 222, 113903), sample pad (Grade 319, 113863) and conjugation pad (Grade 8964, 003171) was purchased from AHLSTROM Co. Ltd., (Helsinki, Finland). Backing card was purchased from PJGo, InC (Seoul, Republic of Korea).

### Carboxyl Latex Bead and Bound Detection Antibody Production

The systems and methods herein increased the affinity of target antigen and antibody. More specifically, Carboxyl Latex Bead instead of gold nanoparticles were chosen and Carboxyl modified latex bead was surface-treated with EDC/NHS for in vitro diagnostic use. The resulting reactive and nonreactive macromolecule made with dye fixed in a covalent bond was fixed on the surface and antibody. The resulting molecule was resistant to heat and pH changes and remained stable and can be preserved for longer periods of time.

Carboxylated PS Latex Particles, as purchased from the Magspher, InC (Pasadena, CA 91107 U.S.A.), was agitated to prevent agglutination and ultrasonic waves are applied according to manufacturer's instructions. (https://www.magsphere.com/Products/Carboxylated-Latex-Particles/carboxylated-latex-particles.html). Microspheres were monodisperse before starting the protocol before prior to no aggregation, as confirmed by microscope.
10. The Carboxylated PS Latex particles were put into 0.1 M MES (PH 6.1) buffer. Ultrasonic waves were applied for uniformization. Centrifugation at 1 2,000 rpm for 20 minutes are applied, after which supernatant was removed a nd washed. This was resuspended in 0.1 M MES (PH 6.1) buffer.
11. These beads were attached by covalent bond to antibodies in a 2-step process: MES buffer was used to activate the beads by mixing with EDC an d Sulfo-NHS. Thirty minutes later, beads were washed and resuspended in MES buffer.
12. 4 mg/ml concentration of #66104 monoclonal antibodies were attached to beads using EDC/NHS coupling in the mixer for 3 hours, centrifuged, an d supernatant was removed.
13. The resulting beads were mixed for 30 minutes in a 0.2% ethanolamin e solution.
14. For the control line: 400 nm Carboxylate modified latex bead was applied at a 4 mg/ml concentration Chicken IgY (Boreda Biotech, Seoul, South Korea) using EDC/NHS coupling. This was mixed in 0.2% ethanolamine solution for 30 minutes.
15. 10% casein was added to a final concentration of 1.5%,
16. The microspheres were put into mixer in room temperature for 1 hour. After 1 hour, the content was centrifuged and the supernatant was removed.
17. Resulting microsphere was conserved in 1% casein 100 mM borate buffer.
18. The concentration of resulting microspheres was chosen after measuring the light absorptance at 660 nm. Final microspheres were preserved at 4° C.

### Manufacturing of Biotin and Secondary Detect Antibody Complex

The second point of the systems and methods herein was to increase the signal generated where Biotin and streptavidin, especially Polystreptavidin, were used. The Fc portion of primary capture antibody was affixed to Biotin and test zone (test line) had Polystreptavidin, such that the resulting signal was amplified when the biotin-primary capture antibody complex reacted with polystreptavidin. Streptavidin had a high affinity to Biotin, especially solid phase streptavidin coating allowed for receiving: protein, peptide, PCR fragments, nucleic acid, hapens, and so forth. The polystreptavidin of the systems and methods herein had an even higher affinity for biotin than streptavidin. Polystreptavidin had 12 Biotin binding sites per molecule which allows for stronger bond and amplified signal. The coating by polystreptavidin had strong affinity, high stability to chemical and heat, high absorption capacity, and low nonspecific binding, thus making it appropriate for coating membranes, beads, biochips, and plastic, etc.

### Biotin-Primary Capture Antibody Manufacture

SARS-COV-2 Omicron S mAb SARS-COV-2 S mAb (Anti-SARS-COV-2 Spike RBD Neutralizing Antibody, Chimeric mAb, Human IgG1 (AM122)) monoclonal antibody was diluted in 50 mM Sodium Phosphate buffer (PH7.5) into 1 mg/ml. NHS-Biotin (20217, Thermo Scientific) was added into monoclonal antibodies offered at 1 mg/mL into a concentration of 10 mM. The final concentration of the biotinized antibodies were measured by A280. Final mole concentration of Biotination was 20 biotin per mole of antibody using the QuantTag Biotin kit (BDK-2000, Vector Laboratories, UK).

### Manufacture of LFA Strip for Device

The strip of the systems and methods herein constituted of sample pad, conjugation pad, nitrocellulose membrane, absorbent pad, and backing card. The uncut sheet was inserted to the cutter to be cut in sizes of 60 mm×4 mm. The cut strip was put together into the cassette as below in the correct direction, as described below.

Backing Card (PJGo InC, Seoul, Korea) was attached into nitrocellulose membrane (Nupore Filtration Systems Pvt. Ltd.). Polystreptavidin 1.5 mg/mL solution was prepared in 50 mM SPB and Goat anti-chicken IgY antibody 1.5 mg/mL solution in 50 mM PBS using the Dispenser system (Zeta Co., Seoul, Korea). The test line was 35 mm above the upper part of nitrocellulose membrane and the control line was 26 mm above the upper part of the nitrocellulose membrane at a speed of 0.8 ul/cm. The cut strip was dried at 37° C. temperature for 3 hours. The absorbent pad (AHLSTROM) was attached so that there was an overlap between the membrane 4 mm on the upper part of backing card. The secondary detection antibody (#66105) was: conjugated into Carboxyl Latex Beads and applied to the conjugation pad at a concentration of 3.4%. The control line antibody complex was applied at the control zone at a concentration of 1.3%. The dried conjugation pad was cut to a size of 300 mm×7 mm after which, the pad was attached to the strip, such that overlap with the membrane was 1.5 mm on the upper part of nitrocellulose membrane. The primary Capture antibody after conjugation with biotin was applied at a concentration of 0.34% and let dry; after cutting into a size of 300 mm×7 mm and attaching, such that there was 3 mm overlap with the latex pad. Finally, the sample pad was cut to a size of 300 mm×13 mm attached, such that there was 3 mm overlap with biotin conjugation pad to finished uncut sheet, as depicted in FIG. 2. The manufacturing process of lateral flow chromatography is further described in FIG. 2.

1. A biosensor device comprising:
a lateral flow assay (LFA), wherein the lateral flow assay is a method in a test strip for using a binder conjugated to specific latex bead and a binder conjugated to biotin; and
polystreptavidin.

2. The biosensor of claim 1, where the lateral flow assay (LFA) comprises:
a sample application pad for applying a sample to the test strip;
a biotin pad comprising at least a first target antibody associated with biotin and a primary binder complex, wherein the first target antibody is used for primary detection;
a conjugation pad comprising at least a second target antibody associated with a binder latex bead, such that a first complex is loaded in a first complex application zone,
a first target antigen is complementary to a portion of a target antigen; and
a nitrocellulose membrane with an absorbent pad and waste pad with a carrier backing card downstream of the nitrocellulose membrane.

3. The biosensor of claim 1, wherein the binder enables detection by specifically binding a target analyte comprising proteins, peptides, glycoproteins, proteoglycans, lipoproteins, ionized metals, metabolic product, genetic material, DNA, RNA, DNA bonding protein, nucleotide probe, DNA binding proteins, pathogens, viruses, viral products, bacteria, bacteria products, low-molecular-weight compounds, hormone receptors, and allergy-associated components comprising antibodies, antigens, aptamer, haptens, antigen proteins, and hormone receptors.

4. The biosensor device of claim 1, wherein the target analyte comprises proteins, peptides, glycoproteins, proteoglycans, lipoproteins, ionized metals, metabolic products, genetic material, DNA, RNA, DNA bonding protein, nucleotide probe, DNA binding proteins, pathogens, viruses, viral products, bacteria, bacteria; products, low-molecular-weight compounds, hormone receptors, and allergy-associated components comprising antibodies, antigens, aptamers, haptens, antigen proteins, and hormone receptors.

5. The biosensor devices of claim 1, wherein the latex beads comprises carboxyl latex beads, aminated carboxyl latex beads, polystylene carboxyl latex beads, silicon carboxyl latex beads, metal carboxyl latex beads, quantum dot carboxyl latex beads, magnetic carboxyl latex beads, carbonated carboxyl latex beads, latex microspheres, fluorescent carboxyl latex beads, and cellulose carboxyl latex beads.

6. The biosensor device of claim 5, wherein the carboxyl latex beads are used for the lateral flow assay, wherein the lateral flow assay comprises a surface coating selected from a group comprising cow plasma albumin, casein, low fat milk, legume-fish derived ingredients, and polyethylene glycol and molecularly similar derivatives of polyethylene glycol.

7. The biosensor device of claim 5, where the carboxyl latex bead is used for the lateral flow assay, wherein the lateral flow assay comprises a functionalized surface coating comprising 1-ethyl-3-(3-dimethylaminopropyl) carbodiimide hydrochloride/N-hydroxy succinimide (EDC HCl/NHS) and molecularly similar equivalents thereof.

8. The biosensor device of claim 7, wherein the carboxyl Latex Beads are modified to have polymers affixed to a surface and a dye or a plurality of dyes, wherein the dye is covalent bonded and stably residing inside the carboxyl beads, thereby the dye is less effected by environmental changes and stable for longer preservation and wherein the plurality of dyes is trapped inside latex microspheres within LFA-based immunochromatography, thereby resulting in a color in the latex microspheres for amplifying a signal from light absorption being is amplified upon detection of a reaction.

9. The biosensor device of claim 8, wherein LFA-based immunochromatography comprises a label mediator, wherein the Label mediator is streptavidin or polystreptavidin which has affinity for biotin, thereby the label mediator is a marker for a biotin-bound binder and target analyte complex.

10. The biosensor device of claim 9, wherein polystreptavidin is a polymer made from streptavidin bound to dextran and a plurality of streptavidin particles, wherein the streptavidin bound to the dextran and the plurality of streptavidin particles are configured to be transposed into a space within a polymer frame; thereby enabling a polystreptavidin effect, wherein the polystreptavidin effect comprises a single polystreptavidin binding to a plurality of biotin, a plurality of detection marker reactions, an amplified resulting signal, and an enhanced detection limit.

11. The biosensor device of claim 8, wherein the LFA-based immunochromatography comprises:
the carboxyl latex bead in combination with the amplified resulting signal of the polystreptavidin effect; and
the streptavidin bound to the dextran and the plurality of streptavidin particles are used for the lateral flow assay comprising enzyme-antibody-antigen complexes incorporated into the polymer frame, wherein enzyme-antibody-antigen complexes comprise labeled enzyme-antibody-antigen complexes reacting with an analyte coming into an inner space that results in detection of the said complexes after which the signal is amplified, and the sensitivity is increased.

12. The biosensor devices of claim 1, wherein biotin is conjugated with the binder at the biotin pad, wherein the biotin pad comprises a test line zone, wherein the test line zone comprises polystreptavidin strongly binds to the target analyte and biotin conjugated to preferably a control line, wherein the control line binds to a tertiary binder configured to detect a presence of the sample, with or without the target analyte, a control zone upstream or downstream of the test zone.

13. The biosensor device of claim 1, wherein the carboxyl Latex beads is modified by EDC/NHS surface treatment in immunochromatography, thereby detecting the target analyte, wherein the target analyte comprises: proteins, peptides, glycoproteins, proteoglycans, lipoproteins, ionized metals, metabolic products, genetic material, nucleic acids, nucleotide probes, DNA binding proteins, pathogens, viruses, viral products, bacteria, and bacteria products low-molecular-weight compounds, hormone receptors, and allergy-associated components comprising antibodies, antigens, aptamers, and haptens.

14. The biosensor device of claim 1; wherein the immunochromatography-based LFA resides within an in vitro diagnostic device comprising a target material and a binder, wherein the target material is an antigen, wherein the binder is an antibody attached to Biotin in the biotin pad and the carboxyl latex beads comprising a detection antibody in the conjugation pad and a signal-inducing complex, wherein the detection antibody in the conjugation pad and the signal-inducing complex is the streptavidin attached to dextran and antigen-antibody complex detected by biotin-polystreptavidin bonds.

15. The biosensor device of claim 1, wherein the immunochromatography-based LFA resides within an in vitro diagnostic device comprise the nitrocellulose membrane in a detection zone comprising:
at least one test zone associated with at least one immobilization agent, wherein the immobilization agent is immobilized in a test zone of the test strip by the biotin-polystreptavidin bond to the Polystreptavidin attached to a dextran polymer spine; and
a sample, a first complex, and a second complex loaded on the test strip at locations such that the sample encounters a lysis zone, and the first complex and the second complex detect a target nucleic acid in the sample, while running an assay preferably containing the test line and polystreptavidin reacts strongly to biotin in a target material-binder complex affixed therein.

16. The biosensor device in claim 1; wherein the immunochromatography-based LFA resides within an in vitro diagnostic device comprise the nitrocellulose membrane in a detection zone comprising:
the test strip preferably including at least one control zone, wherein the at least one control zone is a control line operatively connected to a binder configured react regardless of whether the target antigen exists in the sample as long as a common component is present, wherein the common component is human antigen and the control line includes mouse or goat anti-chicken IgY antibodies as a tertiary antibody.

17. The biosensor device of claim 1, wherein the immunochromatography-based LFA resides within an in vitro diagnostic device configured to diagnose a qualitative and/or semiquantitative presence of a target by interpreting a color associated with streptavidin or Polystreptavidin having affinity for the biotin, thereby forming a marker for the biotin-bound latex microsphere binder and target material complex.

18. The biosensor device of claim 1, wherein the immunochromatography-based LFA resides within an in vitro diagnostic device comprises a piece of uncut sheet cut by the size of 60 mm×4 mm put into a plastic housing or cassette.

19. The biosensor device of claim 1, wherein the immunochromatography-based LFA resides within an in vitro diagnostic device comprises: a polystyrene latex bead; an antibody mediating analyte and marker; and a biotin bound binder, wherein the polystyrene latex bead, the antibody mediating analyte and marker; and the biotin bound binder are operatively connected to detect the presence of the antibody antigen complex by biotin-polystreptavidin binding.

20. The biosensor device of claim 1; wherein the target analyte is a target antigen and the primary complex comprises:
at least one first target antibody associated with biotin, wherein the first target antibody is complementary to a portion of the target antigen;
at least one secondary complex comprising at least one second (secondary detection) target antibody (binder) associated with a binder latex bead wherein the secondary detection antibody is complementary to a portion of the target antigen; and
a sandwich type assay.

21. The biosensor device of claim 1; wherein the latex bead includes:
chicken IgY; and
the control line comprising a coating by mouse or goat anti-chicken IgY antibodies as the tertiary antibody.

22. The biosensor device of claim 1; wherein the LFA is configured for detecting a target, wherein the target is associated with a presence of bacterial infection, a virus, a fungus, a parasite, a malignancy tumor antigen, an autoimmune condition, and a trauma.

23. The biosensor device of claim 22, wherein the bacterial infection, the virus, the fungus, the parasite, the malignancy tumor antigen, the autoimmune condition, and the trauma comprise: Anthrax, Botulism, Cholera, Diphtheria, Influenza, Measles, Meningococcal disease, Middle East Respiratory Syndrome (MERS), Plague, Rabies, human, Rubella (not congenital), Severe acute respiratory syndrome (SARS), Smallpox, Tularemia, Viral hemorrhagic fever (VHF), including Ebola virus disease, Lassa fever, Marburg hemorrhagic fever, and Crimean-Congo hemorrhagic fever, Yellow fever, Arboviral neuroinvasive and non-neuroinvasive disease, Eastern equine encephalitis virus disease, LaCrosse virus disease, California serogroup virus disease, Powassan virus disease, St. Louis encephalitis virus disease, West Nile virus disease, Western equine encephalitis virus disease, Chancroid Cyclosporiasis, Coccidioidomycosis Dengue, E. coli O157: H7, Shiga toxin-producing E. coli Foodborne disease outbreaks, Granuloma inguinale, Haemophilus influenzae, Hantavirus Hemolytic uremic syndrome (HUS), Hepatitis A, Hepatitis B, perinatal Influenza-associated pediatric mortality, Legionnaires' disease, Listeriosis, Lymphogranuloma venereum, Malaria Meningitis, viral meningoencephalitis, Mumps, Pertussis Poliomyelitis, Psittacosis Q fever Rubella (congenital), Salmonellosis, Shigellosis, Staphylococcus aureus with resistance or intermediate resistance to Vancomycin (VRSA, VISA), Syphilis, Tetanus, Tuberculosis, multi-drug resistant tuberculosis (MDR-TB), Typhoid fever, Waterborne disease outbreaks, Amebiasis Botulism, wound Botulism, infant Brucellosis, Campylobacteriosis, Chlamydia infections, urethritis, epididymitis, cervicitis, pelvic inflammatory disease, neonatal conjunctivitis, pneumonia, Creutzfeldt-Jakob disease (CJD), Cryptosporidiosis Cytomegalovirus (CMV), congenital Ehrlichiosis Encephalitis, Encephalitis, postinfection Giardiasis Gonococcal infections, urethritis, cervicitis, pelvic inflammatory disease, pharyngitis, arthritis, endocarditis, meningitis and neonatal conjunctivitis, Hepatitis B, non-perinatal Hepatitis C, Hepatitis D, delta hepatitis, Hepatitis E Herpes, Kawasaki disease, mucocutaneous lymph node syndrome, Leprosy, Hansen disease, Leptospirosis, Lyme disease, Meningitis, Mycobacterial disease other than tuberculosis (MOTT), Reye syndrome, Rheumatic fever, Rocky Mountain spotted fever (RMSF), Streptococcal disease, group A, invasive (IGAS), Streptococcal disease, group B, newborn Streptococcal toxic shock syndrome (STSS), Streptococcus pneumoniae, invasive disease, Toxic shock syndrome (TSS) Toxoplasmosis, Trichinosis, Typhus fever, Varicella, Vibriosis, Yersiniosis, Influenza, Blastomycosis, Conjunctivitis, acute Histoplasmosis, Pediculosis, Scabies Sporotrichosis, Staphylococcal skin infections, and Toxoplasmosis.

24. The biosensor device of claim 22, wherein the target is a tumor marker comprising: alpha-feto protein, beta 2 microglobulin, carcinoembryonic antigen (CEA), CA15-3, CA125, CA19-9, HEA, PSA, CYFRA21-1, neuron specific enolase (NSE), PIVKA-2, and chromogranin A.

25. The biosensor device of claim 1; wherein LFA-based immunochromatography is used to detect SARS-Cov-2 virus (SARS-Cov-2) infection (COVID-19).

26. The biosensor device of claim 1, wherein the target antigen is a nucleocapsid protein (N protein, NP) of SARS-Cov-2 and primary detection and secondary capture specific antibodies made by hybridoma or recombination technology and myeloma cell line used to detect the target antigen.

27. The biosensor device of claim 1, wherein the target antigen is the spike protein (S protein, S) or any other antigen of SARS-Cov-2 and specific antibodies made by hybridoma or recombination technology and myeloma cell line used to detect the target antigen.

28. The biosensor device of claim 1, further comprising commercialized antibodies uses a NP monoclonal antibody.

29. The biosensor device of claim 1; wherein the primary detection and secondary capture specific antibodies, the Goat anti-chicken IgY antibodies, and the NP monoclonal antibody, wherein the NP monoclonal antibody is made for a NP antigen of Sars-COV; SARS-COV-2 NP mAb and SARS-COV-2 NP mAb and wherein the Goat anti-chicken IgY antibodies are used for a control line.

30. The biosensor device of claim 1, where LFA is used in respiratory samples, wherein the respiratory sampling comprising nasopharyngeal swab, oropharyngeal swab, saliva, sputum, and the respiratory samples are mixed with nucleic acid extraction fluid and put into the sample application zone.

31. The biosensor device of claim 1, wherein the LFA comprises a visible line in the detection zone and control line if target antigen is present in the sample due to the SARS-COV-2 NP monoclonal detection antibody and goat anti-chicken IgY antibody in the presence of SARS-COV2 antigen, wherein the target antigen is a NP antigen.

32. The biosensor device of claim 1, wherein LFA comprises a visible control line turning red due to detection of NP antigen in the sample and not the goat anti-chicken IgY antibody in the absence of SARS-COV2 antigen in the sample.

33. The biosensor device of claim 1, wherein the LFA comprises a single test line or multiple test lines, wherein the multiple test lines are configured to detect multiple targets such that presence of each target of the multiple targets corresponds to a separate test line of the multiple test lines such that the presence of multiple targets is indicated on the same test line wherein the multiple targets have different characteristics than a single target, wherein the presence of multiple targets on the same test line is visually indicated by a different color than the presence of each of the targets alone.

34. The biosensor device of claim 1, wherein the LFA device is configured for detecting NP antigen from SARS-COV-2 in the sample comprising of any combination of:
i. a sample application zone for applying the sample to the test strip;
ii. a lysis buffer optionally incorporated to the sample application zone comprising at least one lysis or denaturing agent that lysis the target antigen or nucleic acid from the liquid analyte, wherein the sample application and the lysis buffer optionally incorporated in a zone;
iii. a biotin pad comprising at least one primary complex comprising at least one first primary capture target antibody associated with at least one biotin binder biotin, wherein the target antigen if present in sample is complementary to a portion of the SARS-COV-2 NP monoclonal antibody which is the first primary capture antibody and first antibody-antigen complex loaded into the next conjugation pad;
iv. a latex conjugation pad in which at least one secondary complex comprises at least one secondary detection target antibody associated with the binder latex bead and a secondary SARS-COV-2 NP monoclonal detection antibody complementary to a portion of the target SARS-COV-2 NP antigen
v. a nitrocellulose membrane comprising:
a) at least one detection zone has Polystreptavidin binding to latex in the complex if antigen is present in the complex and is clearly visible with a color
b) at least one control zone comprising the control line, wherein the control zone is:
downstream of the test zone and shows a visible line if sample is present due to anti-goat antibody binding, or
upstream of the test zone
vi. an absorbent pad and waste pad with carrier backing card at downstream of the nitrocellulose membrane

35. The biosensor device of claim 1, wherein the LFA is used for Point of care (POC) testing.

36. The biosensor device of claim 1, wherein the target antigen is the spike protein (S protein, S) of any of the variants of SARS-Cov-2 and specific antibodies made by hybridoma or recombination technology and myeloma cell line used to detect the target antigen.

37. The biosensor device of claim 1, further comprising commercialized antibodies uses a S monoclonal antibody.

38. The biosensor device of claim 1; wherein the primary detection and secondary capture specific antibodies, the Goat anti-chicken IgY antibodies, and the S monoclonal antibody, wherein the S monoclonal antibody is made for a NP antigen of Sars-COV; SARS-COV-2 S mAb and SARS-COV-2 S mAb and wherein the Goat anti-chicken IgY antibodies are used for a control line, by itself or in combination with the above claimed NP ag-Ab technology

39. The biosensor device of claim 1, where LFA is used in respiratory samples, wherein the respiratory sampling comprising nasopharyngeal swab, oropharyngeal swab, saliva, sputum, and the respiratory samples are mixed with nucleic acid extraction fluid and put into the sample application zone.

40. The biosensor device of claim 1, wherein the LFA comprises a visible line in the detection zone and control line if target antigen is present in the sample due to the SARS-COV-2 S monoclonal detection antibody and goat anti-chicken IgY antibody in the presence of SARS-COV2 antigen, wherein the target antigen is a S antigen.

41. The biosensor device of claim 1,
wherein LFA comprises a visible control line turning red due to detection of S antigen in the sample and not the goat anti-chicken IgY antibody in the absence of SARS-COV2 antigen in the sample.

42. The biosensor device of claim 1, wherein the LFA comprises a single test line or multiple test lines, wherein the multiple test lines are configured to detect multiple targets such that presence of each target of the multiple targets corresponds to a separate test line of the multiple test lines such that the presence of multiple targets is indicated on the same test line wherein the multiple targets have different characteristics than a single target, wherein the presence of multiple targets on the same test line is visually indicated by a different color than the presence of each of the targets alone.

43. The biosensor device of claim 1, wherein the LFA device is configured for detecting S antigen from SARS-COV-2 in the sample comprising of any combination of:
i. a sample application zone for applying the sample to the test strip;
ii. a lysis buffer optionally incorporated to the sample application zone comprising at least one lysis or denaturing agent that lysis the target antigen or nucleic acid from the liquid analyte, wherein the sample application and the lysis buffer optionally incorporated in a zone;
iii. a biotin pad comprising at least one primary complex comprising at least one first primary capture target antibody associated with at least one biotin binder biotin, wherein the target antigen if present in sample is complementary to a portion of the SARS-COV-2 S monoclonal antibody which is the first primary capture antibody and first antibody-antigen complex loaded into the next conjugation pad;
iv. a latex conjugation pad in which at least one secondary complex comprises at least one secondary detection target antibody associated with the binder latex bead and a secondary SARS-COV-2 S monoclonal detection antibody complementary to a portion of the target SARS-COV-2 S antigen (There may be 1 additional latex conjugation pad in which comprises at least one secondary detection target antibody associated with the binder latex bead and a secondary SARS-COV-2 NP monoclonal detection antibody complementary to a portion of the target SARS-COV-2 NP antigen as in Example 9-2)
v. a nitrocellulose membrane comprising:
a) at least one detection zone has Polystreptavidin binding to latex in the complex if antigen is present in the complex and is clearly visible with a color
b) at least one control zone comprising the control line, wherein the control zone is:
downstream of the test zone and shows a visible line if sample is present due to anti-goat antibody binding, or
upstream of the test zone
vi. an absorbent pad and waste pad with carrier backing card at downstream of the nitrocellulose membrane

44. The biosensor device of claim 1, wherein the LFA is used for Point of care (POC) testing of SARS-COV-2 and/or its variants.
